(19) United States
(12) Patent Application Publication
(10) Pub. No.: US 2021/0148936 A1
(43) Pub. Date: May 20, 2021

(54) TREATMENTS AND BIOMARKERS FOR THE PROGNOSIS OF ZIKA VIRUS INFECTION

(71) Applicant: Erasmus University Medical Center Rotterdam, Rotterdam (NL)

(72) Inventors: Barry Hubertus Gerardus ROCKX, Houten (NL); Bram Cornelis Jeroen VAN DER EERDEN, Prinsenbeek (NL); Maria Petronella Gerarda KOOPMANS, Huis ter Heide (NL); Johannes Petrus Thomas Maria VAN LEEUWEN, Amstelveen (NL)

(21) Appl. No.: 17/055,558

(22) PCT Filed: May 15, 2019
(86) PCT No.: PCT/NL2019/050283

§ 371 Date: Nov. 13, 2020

(30) Foreign Application Priority Data
May 15, 2018 (EP) 18172463.4

(51) Int. Cl. G01N 33/68

(52) U.S. Cl. CPC G01N 33/6893; G01N 2800/52; G01N 2800/50

(57) ABSTRACT

The disclosure provides methods, biomarkers, and kits for determining the prognosis of Zika virus infection in relation to severity of symptoms resulting from effects of the Zika virus infection on bone metabolism, such as arthralgia, osteoporosis, and craniosynostosis. The disclosure further provides treatments for Zika virus infection, in particular for treating arthralgia, osteoporosis, and craniosynostosis.

### FIELD OF THE INVENTION

The disclosure provides methods, biomarkers, and kits for determining the prognosis of Zika virus infection in relation to severity of symptoms resulting from effects of the Zika virus infection on bone metabolism, such as arthralgia, osteoporosis, and craniosynostosis. The disclosure further provides treatments for Zika virus infection, in particular for treating arthralgia, osteoporosis, and craniosynostosis.

### BACKGROUND OF THE INVENTION

Zika virus (ZIKV) is an arthropod borne (arbo) virus and belongs to the family of Flaviviridae. It has a single-stranded RNA genome that encodes for a single polyprotein. Generally, arboviruses are known to replicate in dendritic cells. It has also been reported that ZIKV infections are highly neurotropic.

Since its discovery in Uganda in 1947, ZIKV has invaded different territories around the world. In 2015, ZIKV was identified in Brazil resulting in an epidemic of unprecedented scale in the Americas and the Caribbean (1). The majority of human ZIKV infections appear to be asymptomatic. However, severe complications due to ZIKV infection are observed during pregnancy, including congenital abnormalities such as microcephaly and subcortical calcifications (3). In adults, acute symptomatic ZIKV infection is typically characterized by a self-limiting disease with fever, rash, conjunctivitis, myalgia and arthralgia/arthritis (4). A rare complication also observed is Guillain-Barre syndrome.

Diagnostic tests for detecting infection by ZIKV are available. However, since many infected individuals will remain asymptomatic, there is a need for tools for predicting the prognosis of infection, in particular for predicting the development of symptoms and/or for predicting the severity of symptoms which will develop. This is particularly relevant when deciding whether to begin medical treatments before an individual has developed symptoms or if only mild symptoms are present. The present disclosure addresses this need.

There is also a current need for new treatments for ZIKV infection, in particular treatments which prevent or reduce arthralgia, osteoporosis, or craniosynostosis. In particular, treatments are needed which are based on the underlying molecular mechanisms that cause such symptoms. The present disclosure addresses this need.

### SUMMARY OF THE INVENTION

In one aspect the disclosure provides methods for determining the prognosis of an individual infected with Zika virus (ZIKV), in particular for predicting complications arising from effects of infection on bone metabolism. Preferably, wherein determining the prognosis comprises predicting the severity of and/or risk of developing one or more symptoms. Preferably, the symptoms are a result of the effects of ZIKV infection on bone metabolism. The methods comprise determining the expression and/or activity of one or more biomarkers indicative of osteoblast and/or osteoclast function in a sample, wherein perturbation of osteoblast and/or osteoclast function indicates a perturbation on bone metabolism and a poor prognosis of said individual. Symptoms resulting from this perturbation include disorders related to reduced mineralization of osteoblasts such as arthralgia and osteoporosis, and bone developmental problems such as craniosynostosis.

In some embodiments, the one or more biomarkers are selected from interferon gamma or one or more markers of the type I interferon signaling pathway, alkaline phosphatase, osteocalcin, the RANKL/OPG ratio, IL-6, IL-1 beta, CTX-I, DPD, one or more markers of the Fibroblast Growth Factor Receptor pathway, one or more markers of the BMP signaling pathway, one or more markers marker of the Wnt signaling pathway.

In some embodiments, the symptom is arthralgia or osteoporosis and the sample is from said individual. In some embodiments the symptom is craniosynostosis and the sample is from said individual or the individual is a fetus and the sample is from a subject carrying the fetus.

In preferred embodiments, the disclosure provides a method for predicting the severity of and/or risk of developing arthralgia or osteoporosis in an individual infected with Zika virus (ZIKV), said method comprising determining the expression and/or activity level of one or more biomarkers indicative of osteoblast and/or osteoclast function in a sample, wherein perturbation of osteoblast and/or osteoclast function indicates poor prognosis of said individual, e.g., that the individual is likely to develop arthralgia or osteoporosis. Preferably, the one or more biomarkers are selected from interferon gamma or one or more markers of the type I interferon signaling pathway, alkaline phosphatase, the RANKL/OPG ratio, IL-6, IL-1 beta, CTX-I, or DPD. More preferably, the one or more biomarkers are selected from interferon gamma, alkaline phosphatase, IL-6, or RANKL/OPG ratio. Preferably the method comprises:
determining the expression of one or more of MX1, OAS1, G1P3, IFIT1, G1P2, IFIT3, IFITM1, IFI35, STAT1, TAP1, IRF9, PSMB8, IFITM3, STAT2, or IRF1;
determining the expression of interferon gamma,
determining the expression of IL-6;
determining the expression of alkaline phosphatase, and/or
determining the RANKL/OPG ratio.

In preferred embodiments, the disclosure provides a method for predicting the severity of and/or risk of developing craniosynostosis in an individual infected with Zika virus (ZIKV), said method comprising determining the expression and/or activity level of one or more biomarkers indicative of osteoblast and/or osteoclast function in a sample, wherein perturbation of osteoblast and/or osteoclast function indicates poor prognosis of said individual, e.g., that the individual is likely to develop Craniosynostosis. Preferably, the one or more biomarkers are selected from alkaline phosphatase, the RANKL/OPG ratio, IL-6, IL-1 beta, CTX-I, DPD, one or more markers of the Fibroblast Growth Factor Receptor pathway, one or more markers of the BMP signaling pathway, or one or more markers marker of the Wnt signaling pathway. More preferably, the one or more biomarkers are selected alkaline phosphatase, the RANKL/OPG ratio, IL-6, one or more markers of the Fibroblast Growth Factor Receptor pathway, one or more markers of the BMP signaling pathway, or one or more markers marker of the Wnt signaling pathway. Preferably, the method comprises:
determining the expression of alkaline phosphatase,
determining the RANKL/OPG ratio;
determining the expression of IL-6;
determining the expression of FGFR2, FGF18, ERF, SPRY1, SPRY2, SPRY4, FGF2, FRS2, FGFR1, FGFR2, FGFRL1, THBS1, FGF5, FGF14, FGFR3, and/or FGF7;
determining the expression of AMH, FST, BMP2, TGIF1, BMP8B, TGIF2, ACVR2B, BMP4, JAG2, INHBB, SMAD7, SMAD6, JAG1, TGFB1I1, NOG, and/or BMP6;
determining the expression of SOS9, SOX4, SOX15, TLE1, SOX6, EP300, CREBBP, FZD8, SFRP2, MYC, TLE3, AXIN1, and/or PP2RC; and/or
determining the expression of CREBBP, FZD8, NFKB2, NFATC2, AXIN1, PLCB4, and/or PLCE1.

In one aspect, the disclosure provides a method for preventing or reducing the effects from perturbation of bone metabolism in an individual infected with Zika virus. In some embodiments, the methods are for treating, e.g., arthralgia, osteoporosis, or craniosynostosis. In some embodiments, the methods comprise administering to an individual in need thereof a composition that alters osteoblast and/or osteoclast function.

In one aspect the disclosure provides a method for treating an individual infected with Zika virus (ZIKV), said method comprising determining the prognosis of said individual as disclosed herein and treating an individual determined to have a poor prognosis with a treatment for arthralgia. Preferably a method is provided for treating an individual infected with Zika virus (ZIKV) for arthralgia, said method comprising determining the expression and/or activity level of one or more biomarkers indicative of osteoblast and/or osteoclast function in a sample, wherein the one or more biomarkers are selected from one or more markers of the type I interferon signaling pathway, interferon gamma, IL-6, alkaline phosphatase, or RANKL/OPG ratio, and treating an individual determined to have a poor prognosis with a treatment for arthralgia.

In one aspect the disclosure provides a method for treating an individual infected with Zika virus (ZIKV), said method comprising determining the prognosis of said individual as disclosed herein and treating an individual determined to have a poor prognosis with a treatment for osteoporosis. Preferably a method is provided for treating an individual infected with Zika virus (ZIKV) for osteoporosis, said method comprising determining the expression and/or activity level of one or more biomarkers indicative of osteoblast and/or osteoclast function in a sample, wherein the one or more biomarkers are selected from one or more markers of the type I interferon signaling pathway, interferon gamma, IL-6, alkaline phosphatase, or RANKL/OPG ratio, and treating an individual determined to have a poor prognosis with a treatment for osteoporosis.

In one aspect the disclosure provides a method for treating an individual infected with Zika virus (ZIKV), said method comprising determining the prognosis of said individual as disclosed herein and treating an individual determined to have a poor prognosis with a treatment for craniosynostosis. Preferably a method is provided for treating an individual infected with Zika virus (ZIKV) for craniosynostosis, said method comprising determining the expression and/or activity level of one or more biomarkers indicative of osteoblast and/or osteoclast function in a sample, wherein the one or more biomarkers are selected from alkaline phosphatase, the RANKL/OPG ratio, IL-6, one or more markers of the Fibroblast Growth Factor Receptor pathway, one or more markers of the BMP signaling pathway, or one or more markers marker of the Wnt signaling pathway and treating an individual determined to have a poor prognosis with a treatment for craniosynostosis.

In preferred embodiments of the methods disclosed herein, the method further comprising obtaining a sample from said individual or, if the individual is a fetus, the sample from a subject carrying the fetus. Preferably, the sample is a bodily fluid.

In some embodiments the disclosure provides kits suitable for determining the prognosis of an individual infected with Zika virus (ZIKV), preferably wherein determining the prognosis comprises predicting the severity of symptoms, said kit comprising at least three of the following:
one or more binding agents that bind IFNγ,
one or more binding agents that bind alkaline phosphatase (ALP) or one or more compounds for determining ALP activity,
one or more binding agents that bind osteocalcin,
one or more binding agents that bind RANKL and one or more binding agents that bind OPG,
one or more binding agents that bind IL-6,
one or more binding agents that bind IL-1beta,
one or more binding agents that bind CTX-I,
one or more binding agents that bind DPD,
one or more binding agents that bind a member of the Fibroblast Growth Factor Receptor pathway, preferably FGFR2,
one or more binding agents that bind a member of the BMP signaling pathway, preferably BMP4, and
one or more binding agents that bind a member of the Wnt signaling pathway.

Preferably, said kit comprises at least three of the following:
one or more binding agents that bind IFNγ or one or more binding agents that binds a member of the type I interferon signaling pathway,
one or more binding agents that bind alkaline phosphatase (ALP) or one or more compounds for determining ALP activity,
one or more binding agents that bind RANKL and one or more binding agents that bind OPG,
one or more binding agents that bind IL-6,
one or more binding agents that bind a member of the Fibroblast Growth Factor Receptor pathway, preferably FGFR2,
one or more binding agents that bind a member of the BMP signaling pathway, preferably BMP4, and
one or more binding agents that bind a member of the Wnt signaling pathway.

In one aspect the disclosure provides a pharmaceutical composition that perturbs osteoblast and/or osteoclast function for use in the treatment of arthralgia or osteoporosis in an individual infected with Zika virus (ZIKV), wherein said composition comprises:
one or more compounds that reduces the expression or activity of IFNγ, preferably wherein the one or more compounds are selected from is a neutralizing antibody against IFNγ and glucocorticoids;
one or more compounds that increases the expression or activity of alkaline phosphatase (ALP), preferably wherein the one or more compounds is an ALP enzyme replacement therapy, more preferably Asfotase alfa;
one or more compounds that reduces the expression or activity of osteocalcin;
one or more compounds that reduces the RANKL/OPG ratio, preferably wherein the one or more compounds is an antibody against RANKL;
one or more compounds that reduces the expression or activity of IL-6, preferably wherein the one or more compounds is an antibody against IL-6;
one or more compounds that reduces the expression or activity of IL-1β; preferably wherein the one or more compounds is an antibody against IL-1ß.
one or more compounds that increases the expression or activity of the Fibroblast Growth Factor Receptor pathway, preferably wherein the one or more compounds is FGF2 or a nucleic acid molecule encoding FGF2;
one or more compounds that increases the expression or activity of the BMP signaling pathway, preferably wherein the one or more compounds is selected from BMP2, BMP4, follistatin, and nucleic acids encoding BMP2, BMP4, and follistatin;
one or more compounds that increases the expression or activity of the Wnt signaling pathway, preferably wherein the one or more compounds is selected from Wnt (preferably Wnt3a or Wnt5), the small molecule CHIR99021, and lithium chloride;
one or more compounds that comprise a bone anabolic therapy, preferably, the bone anabolic therapy comprises providing Sclerostin (SOST) or a nucleic acid molecule encoding Sclerostin; or
one or more osteoclast activity inhibitors, preferably wherein the inhibitor is a bisphosphate.

Preferably said composition comprises:

one or more compounds that reduces the expression or activity of IFNγ, preferably wherein the one or more compounds are selected from is a neutralizing antibody against IFNγ and glucocorticoids;
one or more compounds that reduces the expression or activity of a member of the type I interferon signaling pathway, preferably wherein the compound is a glucocorticoid;
one or more compounds that increases the expression or activity of alkaline phosphatase (ALP), preferably wherein the one or more compounds is an ALP enzyme replacement therapy, more preferably Asfotase alfa;
one or more compounds that reduces the RANKL/OPG ratio, preferably wherein the one or more compounds is an antibody against RANKL;
one or more compounds that reduces the expression or activity of IL-6, preferably wherein the one or more compounds is an antibody against IL-6;
one or more compounds that comprise a bone anabolic therapy, preferably, the bone anabolic therapy comprises providing Sclerostin (SOST) or a nucleic acid molecule encoding Sclerostin; or
one or more osteoclast activity inhibitors, preferably wherein the inhibitor is a bisphosphate. Preferably methods for treating an individual in need thereof are provided by administering a therapeutically effective amount of the pharmaceutical compositions or one or more compounds disclosed herein.

In one aspect the disclosure provides a pharmaceutical composition that perturbs osteoblast and/or osteoclast function for use in the treatment of craniosynostosis in an individual infected with Zika virus (ZIKV), wherein said composition comprises:
one or more compounds that reduces the expression or activity of alkaline phosphatase (ALP)
one or more compounds that reduces the expression or activity of osteocalcin;

one or more compounds that increases the RANKL/OPG ratio; preferably wherein the one or more compounds is RANKL or a nucleic acid encoding RANKL;
one or more compounds that reduces the expression or activity of Fibroblast Growth Factor Receptor pathway, preferably wherein the one or more compounds is a neutralizing FGFR antibody or FGF trap;
one or more compounds that reduces the expression or activity of the BMP signaling pathway, preferably wherein the one or more compounds is a small molecule inhibitor of BMP signaling, preferably selected from K02288, LDN-193189, and dorsomorphin; Noggin, Gremlin 1, Chordin or a nucleic acid molecule encoding Noggin, Gremlin 1, or Chordin;
one or more compounds that reduces the expression or activity of the Wnt, preferably wherein the one or more compounds is Dkk1 or Sclerostin, or a nucleic acid encoding Dkk1 or Sclerostin;
one or more compounds that stimulate bone turnover, preferably wherein the compound is parathyroid hormone (PTH).

Preferably said composition comprises:
one or more compounds that reduces the expression or activity of alkaline phosphatase (ALP)
one or more compounds that increases the RANKL/OPG ratio; preferably wherein the one or more compounds is RANKL or a nucleic acid encoding RANKL;
one or more compounds that reduces the expression or activity of Fibroblast Growth Factor Receptor pathway, preferably wherein the one or more compounds is a neutralizing FGFR antibody or FGF trap;
one or more compounds that reduces the expression or activity of the BMP signaling pathway, preferably wherein the one or more compounds is a small molecule inhibitor of BMP signaling, preferably selected from K02288, LDN-193189, and dorsomorphin; Noggin, Gremlin 1, Chordin or a nucleic acid molecule encoding Noggin, Gremlin 1, or Chordin;
one or more compounds that reduces the expression or activity of the Wnt, preferably wherein the one or more compounds is Dkk1 or Sclerostin, or a nucleic acid encoding Dkk1 or Sclerostin;
one or more compounds that stimulate bone turnover, preferably wherein the compound is parathyroid hormone (PTH). Preferably methods for treating an individual in need thereof are provided by administering a therapeutically effective amount of the pharmaceutical compositions or one or more compounds disclosed herein.

In some embodiments, the treatment comprising determining the prognosis of said individual by a method disclosed herein

### BRIEF DESCRIPTION OF THE DRAWINGS

FIG. 1: Replication of ZIKV in osteoblasts. Culture supernatant was collected at different time points after infection of primary osteoblasts by ZIKV (moi=5). A) Growth curve kinetics of ZIKV infection in osteoblasts from Donor 4266 (closed circles) and Donor 3520 (open squares) during differentiation over the period of 3 weeks. Error bars represent the standard error of mean (S.E.M). B) Representative immunofluorescent images of ZIKV-infected (right panel) and uninfected controls (left panel).

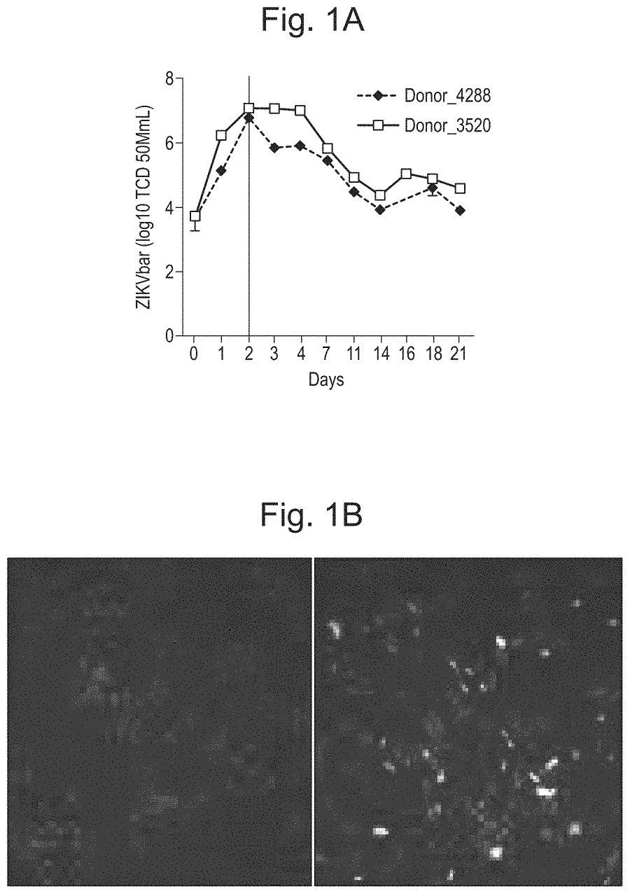

FIG. 2: Effect of ZIKV infection on osteoblast differentiation and maturation. Effect of ZIKV infection on osteoblast differentiation is measured by Alkaline phosphatase (ALP) activity in cultures from A) Donor 4266 and B) Donor 3520, and effect on mineralization is measured as a concentration of calcium present in the cultures from C) Donor 4266 and D) Donor 3520. Results are compared between ZIKV infected (white bars) and uninfected controls (black bars) and ALP levels are normalized against total protein. Error bars represent the standard error of mean (S.E.M).*=p<0.05 Student T-test.

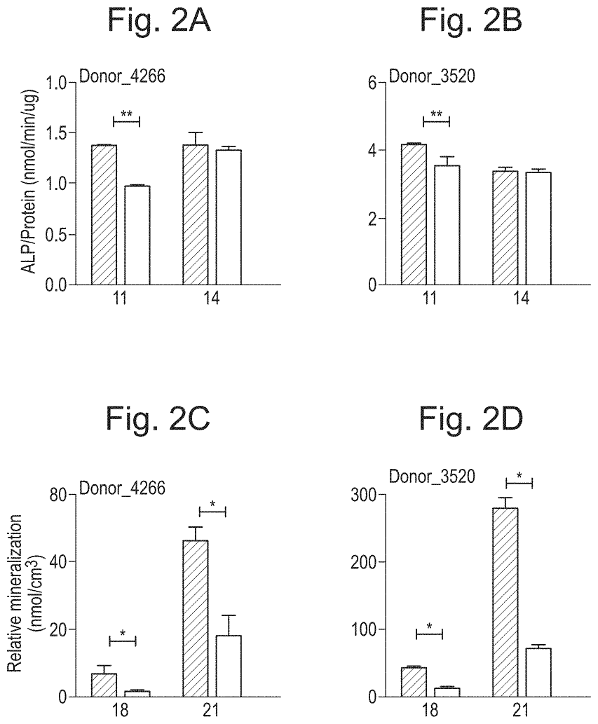

FIG. 3: Gene expression levels of analysed genes after ZIKV infection. Gene expression of key transcription factors from ZIKV infected osteoblasts (white bars) versus uninfected controls (black bars) in (A-C) Donor 4266 and (D-F) Donor 3520. Gene expression was corrected for house keeping gene, GAPDH. Error bars represent the standard error of mean (S.E.M).*=p<0.05 Student T-test.

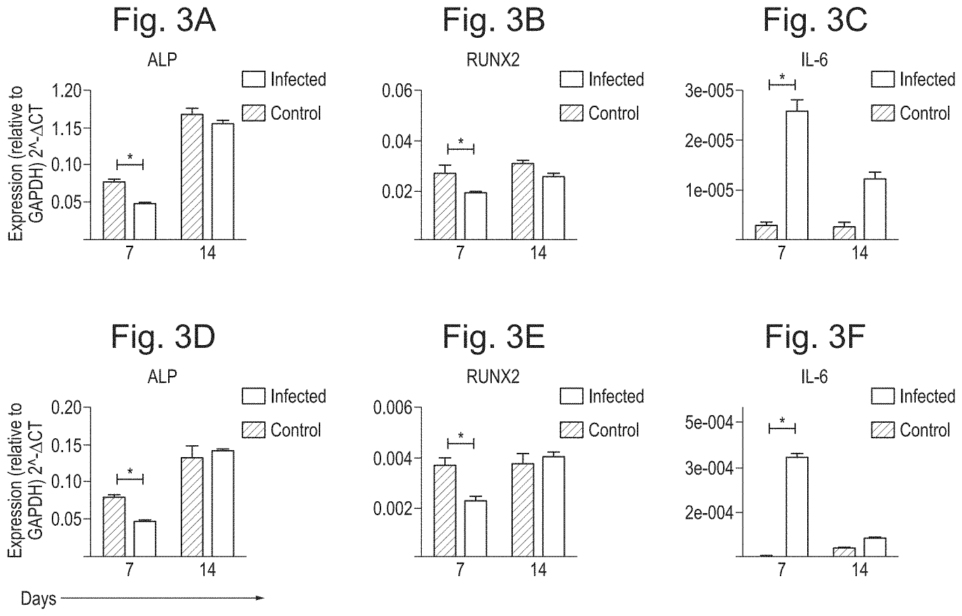

FIG. 4: Gene expression levels of analysed genes after ZIKV infection. Gene expression of RANKL and OPG from ZIKV infected osteoblasts. Gene expression was corrected for house keeping gene, GAPDH. Error bars represent the standard error of mean (S.E.M).

FIG. 5: Gene expression levels of analysed genes after ZIKV infection. Gene expression of FGFR2 from ZIKV infected osteoblasts. Gene expression was corrected for house keeping gene, GAPDH. Error bars represent the standard error of mean (S.E.M).*=p<0.05 Student T-test.

### DETAILED DESCRIPTION OF THE DISCLOSED EMBODIMENTS

The disclosure is based, in part, on the identification of biomarkers that correlate with the prognosis of patients infected with Zika virus (ZIKV). The examples provided herein demonstrate that osteoblasts are affected by ZIKV infection. As described further herein, the examples describe an in vitro model of arthralgia and osteoporosis using human bone marrow-derived mesenchymal stem cells (MSCs). The examples also describe an in vitro model for studying the process of craniosynostosis using human osteoblasts. Bone remodeling is a tightly regulated process, which requires a balance in bone resorption and bone formation by osteoclasts and osteoblasts, respectively (20). Disruption of this balance can lead to abnormal bone remodeling and inception of multiple pathologic conditions. Osteoblasts play a key role in bone remodeling both directly in bone synthesis as well as indirectly by the regulation of bone resorption. Osteoblasts originate from mesenchymal stem cells (MSC). Osteoclasts are multinucleated cells involved in bone resorption. Several inflammatory cytokines such as IL-6 and IL1ß have been associated with arthritis. These key inflammatory mediators have been shown to induce expression of RANKL in cells of the osteoblast lineage. RANK is expressed on osteoclast precursors as well as mature osteoclasts and binding of RANKL induces osteoclast formation and activation, respectively. OPG serves as an decoy receptor by competing with RANK for binding to RANKL. Therefore changes in the ratio of RANKL/OPG can influence the resorption of bone. Induction of RANKL results in an elevated RANKL/OPG ratio and a subsequent increase in osteoclast differentiation and activation.

While not wishing to be bound by theory, a hypothesis is provided herein that ZIKV infection of MSC affects their differentiation into osteoblasts and/or infection of differentiated osteoblasts dysregulates the paracrine function of RANKL and OPG expression through induction of IL-6.

Accordingly, one aspect of the disclosure provides methods for determining the prognosis of an individual infected with Zika virus (ZIKV) comprising determining the expression and/or activity of one or more biomarkers indicative of osteoblast and/or osteoclast function. In some embodiments, the prognosis relates to predicting the effects of ZIKV infection on bone metabolism and symptoms resulting from perturbed bone metabolism. Preferably, the methods disclosed herein are for predicting in a ZIKV infected individual the severity of and/or risk of developing one or more symptoms, including arthralgia, osteoporosis, and craniosynostosis.

Arthralgia results, in part, from pathologic bone loss due to impaired osteoblast function (N. C. Walsh et al., Osteoblast function is compromised at sites of focal bone erosion in inflammatory arthritis. J Bone Miner Res 24, 1572-1585 (2009). Arthralgia/arthritis has been reported in over 70% of ZIKV cases (T. E. Colombo et al., Clinical, laboratory and virological data from suspected ZIKV patients in an endemic arbovirus area. J Clin Virol 96, 20-25 (2017). The majority of these cases are considered mild and self-limiting with arthralgia reported for 3-5 days during acute infection, however persistent or recurrent arthralgia for more than 30 days has been reported (J. F. Chan, et al. J Infect 72, 507-524 (2016). Arthralgia symptoms include stiffness, joint pain, redness, and the reduced ability to move joints. Arthralgia is normally not characterized by joint swelling. In contrast, arthritis is inflammation of a joint and is associated by joint swelling. The methods and biomarkers disclosed herein provide prognostic information in relation to the likelihood that a Zika infected individual will develop arthralgia, and in particular a more severe and/or persistent form of arthralgia.

Osteoporosis is characterized by a decreased density of normally mineralized bone. This results in a weakening of bone and an increased risk of bone fractures and breaks.

Microcephaly is characterized by at least two standard deviation reduction in brain volume, intellectual and motor disabilities, and behavioral issues. This reduction in brain volume results in a reduced head circumference. A dramatic increase in prenatal microcephaly has been observed in ZIKV endemic regions. It has been suggested that ZIKV has the ability to cross the placenta and infect fetal nervous tissues (Sharma and Lal, Frontiers in Microbiology 2017 8:1-14). The long-term consequences of microcephaly depend on underlying brain anomalies and can range from mild developmental delays to severe motor and intellectual deficits, like cerebral palsy.

One factor contributing to microcephaly is craniosynostosis. Craniosynostosis is the premature fusion of one or more of the cranial sutures. Premature osteoblast differentiation in the skull may lead to fusion and early closure of the cranial sutures (craniosynostosis) and results in reduced development of the brain. Evidence of craniosynosthosis has been reported in up to 25% of newborns infected prenatally by ZIKV (M. Del Campo et al., The phenotypic spectrum of congenital Zika syndrome. Am J Med Genet A 173, 841-857 (2017).) Various reports speculate that neurotropism of the virus may play a role in microcephaly. While not wishing to be bound by theory, we hypothesize that premature closure of the cranial structures is a second mechanism by which brain developmental problems can develop, resulting from Zika virus infection of osteoblasts. The methods and biomarkers disclosed herein provide prognostic information in relation to the likelihood that a Zika infected neonate will develop craniosynostosis, and in particular a more severe form of craniosynostosis.

In some embodiments, the biomarkers and methods applying said biomarkers disclosed herein provide an indication as to a perturbation on bone metabolism. While not wishing to be bound by theory, perturbations in bone metabolism can lead to various symptoms. Thus, the biomarkers and methods applying said biomarkers disclosed herein provide an indication as to the risk of developing symptoms and/or an indication of the future severity of said symptoms (i.e., the risk of developing a severe form of the symptoms).

Biomarkers indicative of osteoblast and/or osteoclast function include interferon gamma or one or more markers of the type I interferon signaling pathway, alkaline phosphatase, osteocalcin, the RANKL/OPG ratio, IL-6, IL-1 beta, CTX-I, DPD, a marker of the Fibroblast Growth Factor Receptor pathway, a marker of the BMP signaling pathway, and a marker of the Wnt signaling pathway. In some embodiments, the expression and/or activity of at least two biomarkers is determined. In some embodiments, biomarkers are indicated as interferon gamma or one or more markers of the type I interferon signaling pathway (1), alkaline phosphatase (2), osteocalcin (3), the RANKL/OPG ratio (4), IL-6 (5), IL-1 beta (6), CTX-I (7), DPD (8), a marker of the Fibroblast Growth Factor Receptor pathway (9), a marker of the BMP signaling pathway (10), and a marker of the Wnt signaling pathway (11), wherein the biomarkers comprise 1 and 2; 1 and 3; 1 and 4; 1 and 5; 1 and 6; 1 and 7; 1 and 8; 1 and 9; 1 and 10; or 1 and 11. In some embodiments, the biomarkers comprise 2 and 3; 2 and 4; 2 and 5; 2 and 6; 2 and 7; 2 and 8; 2 and 9; 2 and 10; or 2 and 11. In some embodiments, the biomarkers comprise 3 and 4; 3 and 5; 3 and 6; 2 and 7; 3 and 8; 3 and 9; 3 and 10; or 3 and 11. In some embodiments, the biomarkers comprise 4 and 5; 4 and 6; 4 and 7; 4 and 8; 4 and 9; 4 and 10; or 4 and 11. In some embodiments, the biomarkers comprise 5 and 6; 5 and 7; 5 and 8; 5 and 9; 5 and 10; or 5 and 11. In some embodiments, the biomarkers comprise 6 and 7; 6 and 8; 6 and 9; 6 and 10; or 6 and 11. In some embodiments, the biomarkers comprise 7 and 8; 7 and 9; 7 and 10; or 7 and 11. In some embodiments, the biomarkers comprise 8 and 9; 8 and 10; or 8 and 11. In some embodiments, the biomarkers comprise 9 and 10; or 9 and 11. In some embodiments, the biomarkers comprise 10 and 11.

In some embodiment, at least three, at least four, at least five, at least six, at least 7, at least 8, at least 9, at least 10 or all 11 biomarkers are determined. The use of multiple biomarkers may increase the confidence of the prognosis. Therefore, when multiple markers are used, not all of the biomarkers need to exhibit a significant alteration. It is clear to a skilled person, e.g., that the alteration in expression and/or activity of one of the two tested biomarkers also provides a prognosis (e.g., an indication that the individual will develop a severe form of a symptom).

The type I interferon signaling pathway comprises multiple IFN-alpha subtypes and activation of signaling leads to the transcription of a number of different target genes. Preferred members of the type I interferon signaling pathway include MX1, OAS1, G1P3, IFIT1, G1P2, IFIT3, IFITM1, IFI35, STAT1, TAP1, IRF9, PSMB8, IFITM3, STAT2, and IRF1, more preferably the marker is MX1, OAS1, G1P3, IFIT1, G1P2, or IFIT3. Increased gene expression of these markers is indicative of a poor prognosis in respect to arthralgia and osteoporosis.

IFNγ (interferon gamma) is a soluble cytokine involved in innate and adaptive immunity and is a marker for inflammation. It has also been reported that IFN-γ stimulates osteoclast formation and bone loss (Gao et al. J Clin Invest. 2007 117:122-132). An exemplary human sequence for interferon gamma is as follows:

MKYTSYILAF QLCIVLGSLG CYC QDPYVKE AENLKKYFNA GHSDVADNGT
LFLGILKNWKEESDRKIMQS QIVSFYFKLFKNFKDDQSIQ KSVETIKEDM
NVKFFNSNKK KRDDFEKLTNYSVTDLNVQRKAIHELIQVMAELSPAAKTG
KRKRSQMLFR GRRASQ (signal peptide underlined).

The corresponding mRNA sequence may be found in GenBank as NCBI Reference Sequence: NM_000619.2 (16 Apr. 2018).

Preferably, the level of interferon gamma protein expression is determined. ELISA kits for detecting human IFN-gamma are commercially available (e.g., IFN gamma Human ELISA Kit from Invitrogen™ and Pelikine™ human IFNγ ELISA kit). Flow cytometry based-assays have also been described where intracellular IFN gamma is detected by flow cytometry following cell-permeabilization (Andersson et al., J. Immunol Methods 1988 112:139-142). Methods for measuring IFN-gamma mRNA have also been described (see, e.g., Hein et al. Scand. J. Immunol. 2001 54:285-291).

Alkaline phosphatase (ALP) is a membrane bound glycosylated enzyme that is expressed throughout various tissues (i.e., ALPL: Alkaline Phosphatase, Liver/Bone/Kidney). This enzyme is known to dephosphorylate compounds. The enzyme plays a role in osteoblast differentiation. Mutations in ALPL have been linked to the disorder hypophosphatasia, which is characterized by hypercalcemia and skeletal defects (Mochizuki et al. Eur J Pediatr. 2000 159(5): 375-9).

An exemplary human sequence for ALPL is as follows:

MISPFLVLAIGTCLTNSLVPEKEKDPKYWRDQAQETLKYALELQKLNTNV
AKNVIMFLGDGMGVSTVTAARILKGQLHHNPGEETRLEMDKFPFVALSKT
YNTNAQVPDSAGTATAYLCGVKANEGTVGVSAATERSRCNTTQGNEVTSI
LRWAKDAGKSVGIVTTTRVNHATPSAAYAHSADRDWYSDNEMPPEALSQG
CKDIAYQLMHNIRDIDVIMGGGRKYMYPKNKTDVEYESDEKARGTRLDGL
DLVDTWKSFKPRYKHSHFIWNRTELLTLDPHNVDYLLGLFEPGDMQYELN
RNNVTDPSLSEMVVVAIQILRKNPKGFFLLVEGGRIDHGHHEGKAKQALH
EAVEMDRAIGQAGSLTSSEDTLTWTADHSHVFTFGGYTPRGNSIFGLAPM
LSDTDKKPFTAILYGNGPGYKWGGERENVSMVDYAHNNYQAQSAVPLRHE
THGGEDVAVFSKGPMAHLLHGVHEQNYVPHVMAYAACIGANLGHCAPASS
AGSLAAGPLLLALALYPLSVLF

The corresponding mRNA sequence may be found in GenBank as NCBI Reference Sequence: NM_000478.5 (5 Apr. 2018).

In some embodiments, the level of ALPL protein expression is determined. Preferably, the level of activity is determined. Activity level of Alkaline phosphatase can be determined by a commercially available ALP test (e.g. Mayo medical Laboratories). A related test is the ALP isoenzyme test to measure the amounts of different types of ALP in the blood. This test is usually based on electrophoresis and commercially available (e.g. Mayo medical Laboratories). Various laboratory tests are commercially available, for example Abcam (colorimetric), Sigma Aldrich (Fluorescence), Rockland immunochemicals (ELISA). Kits, especially ELISA kits are available from a broad range of commercial providers.

Osteocalcin is also known as bone gamma-carboxyglutamicacid-containing protein (BGLAP) and is protein hormone found in bone and dentin. This factor is secreted by osteoblasts and regulates bone remodeling and energy metabolism. Osteocalcin is used as a marker for the for the bone formation process, as higher serum-osteocalcin levels are relatively well correlated with increase in bone mineral density.

An exemplary human sequence of osteocalcin is as follows:

MRALTLLALLALAALCIAGQAGAKPSGAESSKGAAFVSKQEGSEVVKRPR
RYLYQWLGAPVPYPDPLEPRREVCELNPDCDELADHIGFQEAYRRFYGPV

The corresponding mRNA sequence may be found in GenBank as NCBI Reference Sequence: NM_199173.5 (1 May 2018).

Preferably, the level of osteocalcin protein expression is determined. The level of osteocalcin can be determined by using an ELISA kit. ELISA kits are commercially available, for example Osteocalcin Human ELISA Kit (ThermoFisher scientific), Human Osteocalcin ELISA Kit (Abeam) and Human Osteocalcin Quantikine ELISA Kit (R&D systems). Furthermore, osteocalcin can be detected histochemically as describes by Vermeulen et al. in 1989 (J Histochem Cytochem).

Receptor activator of nuclear factor kappa-B Ligand (RANKL) is also known as tumor necrosis factor ligand superfamily member 11 (TNFSF11). RANKL is a membrane protein of the tumor necrosis factor superfamily. Its expression is related to the immune system and to control bone metabolism. RANKL functions as a key factor for osteoclast differentiation and activation. Induced RANKL expression leads to increased bone loss.

An exemplary human sequence of RANKL is as follows:

MRRASRDYTKYLRGSEEMGGGPGAPHEGPLHAPPPPAPHQPPAASRSMFV
ALLGLGLGQWCSVALFFYFRAQMDPNRISEDGTHCIYRILRLHENADFQD
TTLESQDTKLIPDSCRRIKQAFQGAVQKELQHIVGSQHIRAEKAMVDGSW
LDLAKRSKLEAQPFAHLTINATDIPSGSHKVSLSSWYHDRGWAKISNMTF
SNGKLIVNQDGFYYLYANICFRHHETSGDLATEYLQLMVYVTKTSIKIPS
SHTLMKGGSTKYWSGNSEFHFYSINVGGFFKLRSGEEISIEVSNPSLLDP
DQDATYFGAFKVRDID

The corresponding mRNA sequence may be found in GenBank as NCBI Reference Sequence: NM_003701.3 (1 Apr. 2018).

OPG is also known as TNF Receptor Superfamily Member 11b (TNFRSF11B). This protein is an osteoblast secreted decoy receptor that functions as a negative regulator of bone resorption. OPG functions as a decoy receptor for RANKL and neutralizes the function of RANKL in bone metabolism.

An exemplary human sequence of OPG is as follows:

MNNLLCCALVFLDISIKWTTQETFPPKYLHYDEETSHQLLCDKCPPGTYL
KQHCTAKWKTVCAPCPDHYYTDSWHTSDECLYCSPVCKELQYVKQECNRT
HNRVCECKEGRYLEIEFCLKHRSCPPGFGWQAGTPERNTVCKRCPDGFFS
NETSSKAPCRKHTNCSVFGLLLTQKGNATHDNICSGNSESTQKCGIDVTL
CEEAFFRFAVPTKFTPNWLSVLVDNLPGTKVNAESVERIKRQHSSQEQTF
QLLKLWKHQNKDQDIVKKIIQDIDLCENSVQRHIGHANLTFEQLRSLMES
LPGKKVGAEDIEKTIKACKPSDQILKLLSLWRIKNGDQDTLKGLMHALKH
SKTYHFPKTVTQSLKKTIRFLHSFTMYKLYQKLFLEMIGNQVQSVKISCL

The corresponding mRNA sequence may be found in GenBank as NCBI Reference Sequence: NM_002546.3 (1 May 2018).

The ratio between OPG and RANKL is important in the regulation of osteogenesis (RANKL/OPG ratio). Preferably the ratio of RANKL/OPG protein expression is determined. The level of RANKL expression can be determined by ELISA. Kits to determine the level of human RANKL are commercially available (Human TNFSF11 ELISA Kit, Abcam; RANKL (total), soluble (human) ELISA kit Enzo Lifesciences).

The level of OPG expression can be determined by ELISA. Kits to determine the level of human OPG are commercially available (Human Osteoprotegerin ELISA Kit, Abcam; TNFRSF11B Human Instant ELISA™ Kit, ThermoFisher Scientific). The outcome of the ELISA assays may be used to calculate the RANKL/OPG ratio.

Interleukin 6 (IL-6) is a mediator of the inflammatory response. IL-6 is a cytokine that functions in inflammation and the maturation of B-cells. The functioning of IL-6 is implicated in a wide variety of inflammation-associated diseases. IL-6 cytokines are described to act on bone cells and have an important role in the skeleton (Blanchard et al. Cytokine Growth Factor Rev. 2009).

An exemplary human sequence of IL-6 is as follows:

MNSFSTSAFGPVAFSLGLLLVLPAAFPAPVPPGEDSKDVAAPHRQPLTSS
ERIDKQIRYILDGISALRKETCNKSNMCESSKEALAENNLNLPKMAEKDG
CFQSGFNEETCLVKIITGLLEFEVYLEYLQNRFESSEEQARAVQMSTKVL
IQFLQKKAKNLDAITTPDPTTNASLLTKLQAQNQWLQDMTTHLILRSFKE
FLQSSLRALRQM

The corresponding mRNA sequence may be found in GenBank as NCBI Reference Sequence: NM_000600.4 (29 Mar. 2018).

Preferably, the level of IL-6 protein expression is determined. ELISA kits for detecting human IL-6 are commercially available (e.g., PeliKine™ compact human IL-6 ELISA kit; Human IL-6 ELISA Kit (Interleukin-6) High Sensitivity, Abcam; IL-6 Human ELISA Kit, ThermoFisher scientific). Furthermore, a recently developed biosensor to detect IL-6 may be used to detect the levels of IL-6 (Huang et al. Procedia Engineering, 2013).

Interleukin-1 beta (IL-1beta) is a cytokine and is produced by activated macrophages as a proprotein, which is processed to its active form by caspase 1. IL1beta is a mediator of the inflammatory response. Furthermore, IL1beta is reported to be involved in bone resorption under pathological and physiological conditions (Lee et al. International Immunology. 2010).

An exemplary human sequence of IL-1Beta is as follows:

MAEVPELASEMMAYYSGNEDDLFFEADGPKQMKCSFQDLDLCPLDGGIQL
RISDHHYSKGFRQAASVVVAMDKLRKMLVPCPQTFQENDLSTFFPFIFEE
EPIFFDTWDNEAYVHDAPVRSLNCTLRDSQQKSLVMSGPYELKALHLQGQ
DMEQQVVFSMSFVQGEESNDKIPVALGLKEKNLYLSCVLKDDKPTLQLES
VDPKNYPKKKMEKRFVFNKIEINNKLEFESAQFPNWYISTSQAENMPVFL
GGTKGGQDITDFTMQFVSS

The corresponding mRNA sequence may be found in GenBank as NCBI Reference Sequence: NM_000576.2 (1 May 2018).

Preferably, the level of IL-1beta protein expression is determined. ELISA kits for detecting human IL-1B are commercially available (e.g., IL-1 beta Human ELISA Kit, ThermoFisher scientific; Human IL-1 beta ELISA Kit, Abeam). Another method to detect the level of IL-1beta protein expression is the Singleplex kit based on fluorescence (IL-1 beta Human Singleplex Kit, Thermo Fisher Scientific).

Carboxy/terminal collagen crosslinks (CTX-1), also known as the C-terminal telopeptide, are degradation products from Type I collagen and are well-known bone resorption biomarkers. ELISA assays for detecting CTX-I are commercially available, such as Serum CrossLaps™, which detects cross-linked EKAHD-ß-GGR sequences.

Deoxypyridinoline (DPD) is a derivative of hydroxypyridinium, which is released during bone resorption into the blood stream and is eliminated unmodified with urine. This molecule is use as a biomarker for bone resorption and can be measured in for example urine or plasma.

Preferably, the level of DPD is determined. ELISA kits to determine the level of DPD are commercially available (e.g., Human DPD (Deoxypyridinoline) ELISA Kit, Elabscience). Alternately, a commercial kit to determine the levels of DPD in urine called MicroVue is available (Quidel). The levels of DPD can also be measured using HPLC, for example in urine.

Fibroblast growth factor receptor pathway influences mitogenesis and differentiation of cells. FGF signaling pathways are also known to be involved in bone development See, Cell Biol Int. 2012 Aug. 1; 36(8):691-6 for a review thereof. One of the major players in this pathway is the Fibroblast growth factor receptor 2 (FGFR2). FGFR2 is a receptor for fibroblast growth factor (FGF).

An exemplary human sequence of FGFR2 is as follows:

MVSWGRFICLVVVTMATLSLARPSFSLVEDTTLEPEEPPTKYQISQPEVY
VAAPGESLEVRCLLKDAAVISWTKDGVHLGPNNRTVLIGEYLQIKGATPR
DSGLYACTASRTVDSETWYFMVNVTDAISSGDDEDDTDGAEDFVSENSNN
KRAPYWTNTEKMEKRLHAVPAANTVKFRCPAGGNPMPTMRWLKNGKEFKQ
EHRIGGYKVRNQHWSLIMESVVPSDKGNYTCVVENEYGSINHTYHLDVVE
RSPHRPILQAGLPANASTWGGDVEFVCKVYSDAQPHIQWIKHVEKNGSKY
GPDGLPYLKVLKAAGVNTTDKEIEVLYIRNVTFEDAGEYTCLAGNSIGIS
FHSAWLTVLPAPGREKEITASPDYLEIAIYCIGVFLIACMVVTVILCRMK
NTTKKPDFSSQPAVHKLTKRIPLRRQVTVSAESSSSMNSNTPLVRITTRL
SSTADTPMLAGVSEYELPEDPKWEFPRDKLTLGKPLGEGCFGQVVMAEAV
GIDKDKPKEAVTVAVKMLKDDATEKDLSDLVSEMEMMKMIGKHKNIINLL
GACTQDGPLYVIVEYASKGNLREYLRARRPPGMEYSYDINRVPEEQMTFK
DLVSCTYQLARGMEYLASQKCIHRDLAARNVLVTENNVMKIADFGLARDI
NNIDYYKKTTNGRLPVKWMAPEALFDRVYTHQSDVWSFGVLMWEIFTLGG
SPYPGIPVEELFKLLKEGHRMDKPANCTNELYMMMRDCWHAVPSQRPTFK
QLVEDLDRILTLTTNEEYLDLSQPLEQYSPSYPDTRSSCSSGDDSVFSPD
PMPYEPCLPQYPHINGSVKT

The corresponding mRNA sequence may be found in GenBank as NCBI Reference Sequence: NM_000141.4 (10 Apr. 2018).

Preferably, the level of Fibroblast growth factor receptor pathway activity is determined. Preferably, the level FGFR2 protein expression is determined. ELISA kits to determine the level of FGFR2 expression are commercially available (e.g., Fibroblast Growth Factor Receptor 2 ELISA Kit, Sigma Aldrich; FGFR2 (Human) ELISA Kit, BioVision). Furthermore, antibodies to detect FGFR2 are commercially available (e.g., Anti-FGFR2 antibody (ab58201), Abcam). A general FGF ELISA kit may be used to determine the pathway activity and is commercially available (Human FGF basic Quantikine ELISA Kit, R&D systems).

Other members of the Fibroblast growth factor receptor pathway useful in the invention disclosed herein include FGF18, ERF, SPRY1, SPRY2, SPRY4, FGF2, FRS2, FGFR1, FGFR2, FGFRL1, THBS1, FGF5, FGF14, FGFR3, and FGF7. Preferably, one or more markers of the Fibroblast Growth Factor Receptor pathway are selected from FGF18 and ERF. Increased gene expression of these markers is indicative of a poor prognosis in respect to Craniosynostosis.

Bone morphogenic proteins (BMP) represent a group of growth factors. These factors are originally discovered by their ability to induce the formation of bone and cartilage. The BMP signaling pathway plays a role in cartilage and bone formation.

Preferably, the level of BMP signaling pathway activation is determined. The level of the BMP signaling pathway can be determined by a BMP4 bioassay kit. ELISA kits to determine the levels of BMP4 are commercially available (e.g., BMP-4 Human ELISA Kit, Thermo Fisher Scientific; Human BMP-4 Quantikine ELISA Kit, R&D systems). The level of BMP signaling pathway activation may be determined by a bioassay for the simultaneous measurement of multiple bone morphogenetic proteins for examples as described by Herrera et al. (BMC Cell Biol. 2009). Other members of the BMP signaling pathway useful in the invention disclosed herein include AMH, FST, BMP2, TGIF1, BMP8B, TGIF2, ACVR2B, BMP4, JAG2, INHBB, SMAD7, SMAD6, JAG1, TGFB1I1, NOG, and BMP6. Preferably, one or more markers of the BMP signaling pathway are selected from AMH, FST, and BMP2. Increased gene expression of these markers is indicative of a poor prognosis in respect to Craniosynostosis.

The Wnt/ß-catenin signaling pathway is known to be involved in embryonic development and adult tissue homeostasis. Furthermore, Wnt signals are key factors to maintain stem cell populations. The role of Wnt signaling in the regulation of bone mass is discussed in the review of Krishnan et al. (J Clin Invest. 2006).

Preferably, the level of Wnt pathway activation is determined. The level of Wnt pathway activity in cells and tissues may be measured by a Wnt/b-catenin reporter assay, such as a luciferase reporter assay responsive to the levels of ß-catenin. Wnt reporter kits are commercially available (e.g., LEADING LIGHT® Wnt Reporter Assay Starter Kit, Enzo Life Sciences). The expression of the protein ß-catenin, the effector protein of the Wnt signaling pathway, may be measured by western blot or immuno-histochemical staining. Antibodies against ß-catenin are commercially available (e.g., Anti-beta Catenin antibody (ab16051) Abcam, 8-Catenin Antibody #9562 CellSignaling). The level of Wnt pathway activation may be determined by analysis of the expression of Axin2, a downstream target gene. Antibodies specific for Axin2 are commercially available (e.g., Anti-Axin 2 antibody (ab32197) Abcam). Other members of the Wnt/ß-catenin signaling pathway useful in the invention disclosed herein include SOS9, SOX4, SOX15, TLE1, SOX6, EP300, CREBBP, FZD8, SFRP2, MYC, TLE3, AXIN1, and PP2RC. Preferably, one or more markers are selected from SOX9 and SOX4. Increased gene expression of these markers is indicative of a poor prognosis in respect to Craniosynostosis.

Members of the Wnt/Ca2+ signaling pathway are also useful in the present invention. Preferred members of this signaling pathway include CREBBP, FZD8, NFKB2, NFATC2, AXIN1, PLCB4, and PLCE1. Increased gene expression of these markers is indicative of a poor prognosis in respect to Craniosynostosis.

Methods are provided wherein the perturbation of osteoblast and/or osteoclast function indicates poor prognosis. Perturbation of function can be identified by measuring the expression and/or activity level of one or more biomarkers. For some of the biomarkers disclosed herein, high expression/activity level indicates poor prognosis whereas for other biomarkers low expression/activity level indicates poor prognosis. The terms “high” and “low” are relative terms. However, a skilled person will recognize that for biomarkers where high expression/activity level indicates poor prognosis, high expression/activity level refers to levels which, e.g., are significantly increased over levels observed in subjects which do not have a poor prognosis (e.g., healthy subjects or subjects which develop mild symptoms) and/or are similar to (or decreased) over levels observed in subjects which have a poor prognosis. Conversely, a skilled person will recognize that for biomarkers where low expression/activity level indicates poor prognosis, low expression/activity level refers to levels which, e.g., are significantly decreased over levels observed in subjects which do not have a poor prognosis (e.g., healthy subjects or subjects which develop mild symptoms) and/or are similar to (or increased) over levels observed in subjects which have a poor prognosis. In exemplary embodiments, the expression and/or activity of one or more of the biomarkers from a sample is compared to a reference value. As is clear to a skilled person, the reference value does not need to be determined separately for each individual. In some embodiments, a value can be determined initially and used as a comparison for later tests. In some embodiments, the reference value may be the biomarker expression/activity level determined in a comparable sample (e.g., from the same type of tissue as the tested tissue, such as blood or serum), from a healthy, i.e., ZIKV negative, (preferably age-matched) individual. In addition, a pool or population of healthy subjects can be used and the reference value can be an average or mean of the measurements from the population. Such a reference value is referred to herein as a good prognosis reference value, since healthy subjects (on average) do not have perturbations in bone metabolism and are not at risk for complications due to such perturbations. In some embodiments, the reference value is from a ZIKV positive (preferably age-matched) individual or a pool or population of ZIKV positive subjects. In some embodiments, the reference value is from a ZIKV positive (preferably age-matched) individual or a pool or population of ZIKV positive subjects that develop a particular symptom or develop a particular symptom severity. For example, expression/activity levels can be measured in ZIKV positive subjects. The development of symptoms and disease progression can be monitored and the subjects can then be stratified based on symptom development/severity. A reference value can be determined which is linked to the expression/activity levels of subjects that later develop mild symptoms. Such a reference value is referred to herein as a good prognosis reference value. A reference value can be determined which is linked to the expression/activity levels of subjects that later develop, e.g., severe symptoms. Such a reference value is referred to herein as a poor prognosis reference value.

In some embodiments, biomarkers are provided, wherein an increase in expression/activity indicates poor prognosis. A significant increase in the expression and/or activity level of a biomarker in the sample as compared to a good prognosis reference value (e.g., value obtained from healthy subjects), or decrease or similar level as compared to a poor prognosis reference value (e.g., value obtained from ZIKV infected subjects who later developed severe symptoms) indicates that the individual has a poor prognosis. In some embodiments, biomarkers are provided, wherein a decrease in expression/activity indicates poor prognosis. A significant decrease in the expression and/or activity level of a biomarker in the sample as compared to a good prognosis reference value, or increase or similar level as compared to a poor prognosis reference value, indicates that the individual has a poor prognosis.

In some embodiments, the symptom is craniosynostosis. While severe complications have been observed in the case of ZIKV infection during pregnancy, not all affected infants will exhibit craniosynostosis, nor will all infants exhibit the same level of craniosynostosis. The biomarkers disclosed herein are useful in methods to determine the risk that a ZIKV infected fetus will develop craniosynostosis. It is understood to a skilled person that being at risk of developing craniosynostosis indicates that the individual has a higher risk of developing craniosynostosis than an age matched population of individuals, or rather the individual has an increased risk over the average of developing craniosynostosis. The biomarkers disclosed herein are also useful in methods to determine the risk that a ZIKV infected fetus will develop a severe form of craniosynostosis. A skilled person is well aware of the differences in severity of craniosynostosis. For example, milder forms of craniosynostosis involve the premature closing of only one of the four sutures.

In some embodiments, prognosis refers to the risk that a fetus will develop craniosynostosis. In some embodiments, biomarkers are provided, wherein a decrease in expression/activity indicates poor prognosis. In an exemplary embodiment, a significant decrease in the expression and/or activity level of such a biomarker in the sample as compared to a good prognosis reference value or a significant increase or similar level as compared to a poor prognosis reference value, indicates that the individual has or is at risk of developing craniosynostosis. Early detection/prediction is important as craniosynosthosis as, when detected timely, it can be treated. In some embodiments, biomarkers are provided, wherein an increase in expression/activity indicates poor prognosis. In an exemplary embodiment, a significant increase in the expression and/or activity level of such a biomarker in the sample as compared to a good prognosis reference value or a significant decrease or similar level as compared to a poor prognosis reference value, indicates that the individual has or is at risk of developing craniosynostosis. In some embodiments, the good prognosis reference value is from a ZIKV negative individual or pool of individuals or from a ZIKV positive individual or pool of individuals that do not develop craniosynostosis. In some embodiments, the poor prognosis reference value is from a ZIKV positive individual or pool of individuals that develop craniosynostosis.

In some embodiments, prognosis refers to the severity of craniosynostosis that a ZIKV infected fetus will develop. In exemplary embodiments, the expression and/or activity of one or more of the biomarkers from a sample is compared to a reference value. In some embodiments, biomarkers are provided, wherein an increase in expression/activity indicates poor prognosis. In an exemplary embodiment, a significant increase in the expression and/or activity level of such a biomarker in the sample as compared to a good prognosis reference value or a significant decrease or similar level as compared to a poor prognosis reference value, indicates that the individual has is at risk for developing a severe form of craniosynostosis. In some embodiments, biomarkers are provided, wherein a decrease in expression/activity indicates poor prognosis. In an exemplary embodiment, a significant decrease in the expression and/or activity level of such a biomarker in the sample as compared to a good prognosis reference value or a significant increase or similar level as compared to a poor prognosis reference value, indicates that the individual is at risk for developing a severe form of craniosynostosis. In some embodiments, the good prognosis reference value is from a ZIKV negative individual or pool of individuals or from a ZIKV positive individual or pool of individuals that develop no or only mild symptoms of craniosynostosis. In some embodiments, the poor prognosis reference value is from a ZIKV positive individual or pool of individuals that develop severe craniosynostosis.

Preferably, the one or more biomarkers for predicting the risk of developing are the severity of craniosynostosis are selected from:
IFNγ, and preferably, decreased expression and/or activity as compared to a good prognosis reference value or increased or no change in expression and/or activity as compared to a poor prognosis reference value indicates poor prognosis;
alkaline phosphatase (ALP), and preferably, increased expression and/or activity as compared to a good prognosis reference value or decreased or no change in expression and/or activity as compared to a poor prognosis reference value indicates poor prognosis;
osteocalcin, and preferably, increased expression and/or activity as compared to a good prognosis reference value or decreased or no change in expression and/or activity as compared to a poor prognosis reference value indicates poor prognosis;
the RANKL/OPG ratio, and an decreased ratio as compared to a good prognosis reference value or increased or no change in expression and/or activity as compared to a poor prognosis reference value indicates poor prognosis;
IL-6, and preferably, decreased expression and/or activity as compared to a good prognosis reference value or increased or no change in expression and/or activity as compared to a poor prognosis reference value indicates poor prognosis;
IL-1beta, and preferably, decreased expression and/or activity as compared to a good prognosis reference value or increased or no change in expression and/or activity as compared to a poor prognosis reference value indicates poor prognosis;
CTX-I, and preferably, decreased expression and/or activity as compared to a good prognosis reference value or increased or no change in expression and/or activity as compared to a poor prognosis reference value indicates poor prognosis;
DPD, and preferably, decreased expression and/or activity as compared to a good prognosis reference value or increased or no change in expression and/or activity as compared to a poor prognosis reference value indicates poor prognosis;
a marker of the Fibroblast Growth Factor Receptor pathway, preferably FGFR2, or selected from FGFR2, FGF18, and ERF, and preferably, increased expression and/or activity as compared to a good prognosis reference value or decreased or no change in expression and/or activity as compared to a poor prognosis reference value indicates poor prognosis;
a marker of the BMP signaling pathway, preferably BMP4, or selected from AMH, FST, or BMP2, most preferably AMH, and preferably, increased expression and/or activity as compared to a good prognosis reference value or decreased or no change in expression and/or activity as compared to a poor prognosis reference value indicates poor prognosis; and
a marker of the Wnt signaling pathway, preferably selected from SOX9, SOX4, and SOX15, and preferably, increased expression and/or activity as compared to a good prognosis reference value or decreased or no change in expression and/or activity as compared to a poor prognosis reference value indicates poor prognosis.

In some embodiments, prognosis refers to the severity of arthralgia or osteoporosis that a ZIKV infected individual will develop. In some embodiments, biomarkers are provided, wherein a decrease in expression/activity indicates poor prognosis. In an exemplary embodiment, a significant decrease in the expression and/or activity level of such a biomarker in the sample as compared to a good prognosis reference value or a significant increase or similar level as compared to a poor prognosis reference value, indicates that the individual is at risk of developing a severe form of arthralgia or osteoporosis. In some embodiments, biomarkers are provided, wherein an increase in expression/activity indicates poor prognosis. In an exemplary embodiment, a significant increase in the expression and/or activity level of such a biomarker in the sample as compared to a good prognosis reference value or a significant increase or similar level as compared to a poor prognosis reference value, indicates that the individual is at risk of developing a severe form of arthralgia or osteoporosis. In some embodiments, the good prognosis reference value is from a ZIKV negative individual or pool of individuals or from a ZIKV positive individual or pool of individuals that develop no or only mild symptoms of arthralgia or osteoporosis. In some embodiments, the poor prognosis reference value is from a ZIKV positive individual or pool of individuals that develop severe arthralgia or osteoporosis.

Preferably, the one or more biomarkers for predicting the severity of arthralgia or osteoporosis are selected from:
IFNγ, and preferably, increased expression and/or activity as compared to a good prognosis reference value or decreased or no change in expression and/or activity as compared to a poor prognosis reference value indicates poor prognosis;
a marker of the type I interferon signaling pathway, preferably selected from MX1, OAS1, G1P3, IFIT1, G1P2, and IFIT3 and preferably, increased expression and/or activity as compared to a good prognosis reference value or decreased or no change in expression and/or activity as compared to a poor prognosis reference value indicates poor prognosis;
alkaline phosphatase (ALP), and preferably, decreased expression and/or activity as compared to a good prognosis reference value or increased or no change in expression and/or activity as compared to a poor prognosis reference value indicates poor prognosis;
osteocalcin, and preferably, decreased expression and/or activity as compared to a good prognosis reference value or increased or no change in expression and/or activity as compared to a poor prognosis reference value indicates poor prognosis;
the RANKL/OPG ratio, and an increased ratio as compared to a good prognosis reference value or decreased or no change in expression and/or activity as compared to a poor prognosis reference value indicates poor prognosis;
IL-6, and preferably, increased expression and/or activity as compared to a good prognosis reference value or decreased or no change in expression and/or activity as compared to a poor prognosis reference value indicates poor prognosis;
IL-1beta, and preferably, increased expression and/or activity as compared to a good prognosis reference value or decreased or no change in expression and/or activity as compared to a poor prognosis reference value indicates poor prognosis;
CTX-I, and preferably, increased expression and/or activity as compared to a good prognosis reference value or decreased or no change in expression and/or activity as compared to a poor prognosis reference value indicates poor prognosis;
DPD, and preferably, increased expression and/or activity as compared to a good prognosis reference value or decreased or no change in expression and/or activity as compared to a poor prognosis reference value indicates poor prognosis;
a marker of the Fibroblast Growth Factor Receptor pathway, preferably FGFR2, and preferably, decreased expression and/or activity as compared to a good prognosis reference value or increased or no change in expression and/or activity as compared to a poor prognosis reference value indicates poor prognosis;
a marker of the BMP signaling pathway, preferably BMP4, and preferably, decreased expression and/or activity as compared to a good prognosis reference value or increased or no change in expression and/or activity as compared to a poor prognosis reference value indicates poor prognosis; and
a marker of the Wnt signaling pathway, and preferably, decreased expression and/or activity as compared to a good prognosis reference value or increased or no change in expression and/or activity as compared to a poor prognosis reference value indicates poor prognosis.

The methods and uses disclosed herein are useful for determining the prognosis of an individual infected with ZIKV. At least two geographically distinct lineages of ZIKV have been reported, namely the African, and the Asian strain. Diagnostic methods for detecting infection by Zika virus are known to a skilled person and include, e.g., the detection of Zika viral RNA in a sample the detection of antibodies produced by an infected individual against the Zika virus. See Sharma and Lal, Frontiers in Microbiology 2017 8:110 and Singh et al. Frontiers in Microbiology 2018: 2677 for a review of ZIKV diagnosis and transmission. In some embodiments, the methods described herein include a step of determining that an individual is infected with ZIKV. When the individual is a neonate, such methods include determining whether the mother and/or neonate is infected with ZIKV. As understood by a skilled person, the methods described herein do not require that a definitive diagnosis of ZIKV infection must be established. Rather, such methods may be carried out if an individual is suspected of ZIKV infection based on, e.g., early symptoms or contact with infected individuals. Thus, “individuals infected with ZIKV” as used herein also includes individuals suspected of being infected by ZIKV.

The expression and/or activity of one or more biomarkers is determined from a sample (i.e., a biological samples). Preferably, expression and/or activity is determined in vitro. In some embodiments, the sample is a biological fluid (e.g., blood, urine, saliva, cerebral spinal fluid (CSF), synovial fluid, semen, lymphatic fluid, amniotic fluid).

The methods described herein relate to determining the expression and/or activity of one or more biomarkers. It is not necessary to determine the absolute amount of biomarker protein in a sample. Rather, as understood by one of skill in the art, the expression and/or activity level of biomarker may be compared between an individual and a reference value and therefore a relative expression level may be determined.

In some embodiments, the expression level of biomarker refers to determining the protein level of the biomarker in a sample. In some embodiments, a binding agent is used to bind and detect a biomarker disclosed herein. Preferably, the step of determining the level of a biomarker can also be expressed as determining the amount of binding of a binding agent to said biological sample from an individual. Binding agents include antibodies as well as non-immunoglobulin binding agents, such as phage display-derived peptide binders, and antibody mimics, e.g., affibodies, tetranectins (CTLDs), adnectins (monobodies), anticalins, DARPins (ankyrins), avimers, iMabs, microbodies, peptide aptamers, Kunitz domains, aptamers and affilins.

In some embodiments, protein expression is determined in an immunoassay. Suitable immunoassays include, e.g., radio-immunoassay, ELISA (enzyme-linked immunosorbant assay), “sandwich” immunoassay, immunoradiometric assay, gel diffusion precipitation reaction, immunodiffusion assay, precipitation reaction, agglutination assay (e.g., gel agglutination assay, hemagglutination assay, etc.), complement fixation assay, immunofluorescence assay, protein A assay, and immunoelectrophoresis assay. In addition to the use of antibody based assays, assays using other biomarker binding compounds may be used. For example, a biomarker binding peptide can be immobilized on a solid support such as a chip. A biological sample is passed over the solid support. Bound biomarker is then detected using any suitable method, such as surface plasmon resonance (SPR) (See e.g., WO 90/05305, herein incorporated by reference).

Antibodies which bind a particular epitope can be generated by methods known in the art. For example, polyclonal antibodies can be made by the conventional method of immunizing a mammal (e.g., rabbits, mice, rats, sheep, goats). Polyclonal antibodies are then contained in the sera of the immunized animals and can be isolated using standard procedures (e.g., affinity chromatography, immunoprecipitation, size exclusion chromatography, and ion exchange chromatography). Monoclonal antibodies can be made by the conventional method of immunization of a mammal, followed by isolation of plasma B cells producing the monoclonal antibodies of interest and fusion with a myeloma cell (see, e.g., Mishell, B. B., et al., Selected Methods In Cellular Immunology, (W.H. Freeman, ed.) San Francisco (1980)).

In some embodiments, protein expression is determined including the detection of peptide fragments of said biomarkers. Peptide fragments are understood as being from 10 to 100 amino acids in length, preferably from 10 to 50 amino acids in length. Peptide fragments can be detected by, e.g. mass spectroscopy. Mass spectroscopy allows detection and quantification of an analyte by virtue of its molecular weight.

Determining the level of expression also includes determining expression of nucleic acid. Preferably, the nucleic acid is RNA, such as mRNA or pre-mRNA. As is understood by a skilled person, the level of RNA expression determined may be detected directly or it may be determined indirectly, for example, by first generating cDNA and/or by amplifying the RNA/cDNA. In some embodiments, nucleic acid is purified from the sample. The level of nucleic acid expression may be determined by any method known in the art including RT-PCR, quantitative PCR, Northern blotting, gene sequencing, in particular RNA sequencing, and gene expression profiling techniques. In some embodiments, the level of nucleic acid is determined using a microarray.

In some embodiments, determining the biomarker expression level further comprises normalizing the expression level of said biomarker. Such methods are well-known to a skilled person and will depend upon the method of protein/nucleic acid measurement used. Typically, the expression of a reference protein/mRNA is determined in parallel to biomarker expression. The normalized expression level of said biomarker may be determined as the ratio of the biomarker to the reference protein. Preferred reference proteins include housekeeping proteins such as actin, beta-tubulin, and

Glyceraldehyde 3-phosphate dehydrogenase (GAPDH). Normalization can also be performed by other known methods such a by normalizing expression level by total cell number. Preferably, both the expression level from the individual and the reference value are normalized to allow for direct comparison.

The disclosure also provides kits which can be used in the methods described herein. In particular, said kits are useful for determining the prognosis of an individual infected with Zika virus (ZIKV). Preferably the kits are useful for predicting the effects of ZIKV infection on bone metabolism and symptoms resulting from perturbed bone metabolism. Preferably, the kits are useful for determining the prognosis comprises predicting the severity of symptoms (e.g., arthralgia, osteoporosis, or craniosynostosis). In some embodiments the kits comprise primer pairs for amplifying the markers disclosed herein. Preferably, the kits comprise primer pairs for amplifying at least one, at least two, or at least three of the markers disclosed herein. In some embodiments, the kits further comprising one or more of the following: DNA polymerase, deoxynucleoside triphosphates, buffer, and Mg2+. In some embodiments the kits comprise control cDNA, preferably the cDNA spans at least one intron-exon boundary such that said primer pair is able to distinguish cDNA from genomic DNA.

In some embodiments, pharmaceutical compositions, uses of said compositions and methods are providing for treating an individual infected with ZIKV. In some embodiments, the compounds disclosed herein may be used to manufacture a medicament used for treating an individual infected with ZIKV.

In some embodiments, said individual is exhibiting no or only mild symptoms of arthralgia or osteoporosis. While not wishing to be bound by theory, the disclosure provides that the perturbation of osteoblast and/or osteoclast function contributes to the development and severity of arthralgia or osteoporosis. In some embodiments, methods are provided comprising treating an individual or determining a treatment regime based on the prognosis determined by the methods disclosed herein. In an exemplary embodiment, the prognosis of an individual infected with ZIKV is determined, e.g., by using one or more biomarkers and disclosed herein. An individual that is prognosed as having a risk of developing a severe form of arthralgia may be treated before exhibiting symptoms (or significant symptoms) of arthralgia. Similarly, an individual that is prognosed as having a risk of developing a severe form of osteoporosis may be treated before exhibiting symptoms (or significant symptoms) of osteoporosis. In this regard, treatment of arthralgia or osteoporosis also refers to the prevention of developing arthralgia/osteoporosis or the prevention of developing a severe form of arthralgia/osteoporosis, or the reduction in the speed of progression of arthralgia/osteoporosis. According, the disclosure provides methods for treating an individual infected with ZIKV for arthralgia or osteoporosis, comprising first determining whether said individual is likely to develop arthralgia/osteoporosis or a severe form of arthralgia/osteoporosis. Since not all individuals infected with ZIKV will develop arthralgia or osteoporosis, the methods allow for personalized, directed treatment only to individuals in need thereof. This allows for earlier treatment and avoids “over-treating” individuals that do not need such treatment.

Additionally, a skilled person is aware of risk factors of osteoporosis (e.g., increased age, being a woman, having a family history of osteoporosis, use of medicines that cause bone thinning, women in menopause or otherwise have low estrogen levels) which can provide further information for determining a prognosis.

In some embodiments, the methods and compositions are useful for treating a fetus. While not wishing to be bound by theory, the disclosure provides that the perturbation of osteoblast and/or osteoclast function contributes to the development and severity of craniosynostosis as a cause of microcephaly. In some embodiments, methods are provided comprising treating an individual or determining a treatment regime based on the prognosis determined by the methods disclosed herein. In an exemplary embodiment, the prognosis of a fetus infected with ZIKV is determined, e.g., by using one or more biomarkers and disclosed herein. A fetus that is prognosed as having a risk of developing craniosynostosis or a severe form of craniosynostosis may be treated before exhibiting symptoms (or significant symptoms) of craniosynostosis or may be treated more aggressively. In this regard, treatment of craniosynostosis also refers to the prevention of developing craniosynostosis or the prevention of developing a severe form of craniosynostosis, or the reduction in the speed of progression of craniosynostosis. According, the disclosure provides methods for treating an individual infected with ZIKV for craniosynostosis, comprising first determining whether said individual is likely to develop craniosynostosis or a severe form of craniosynostosis. Since not all individuals infected with ZIKV will develop craniosynostosis, the methods allow for personalized, directed treatment only to individuals in need thereof. This allows for earlier treatment and avoids “over-treating” individuals that do not need such treatment.

The disclosure also provides novel treatments. In particular, such treatments are useful for altering osteoblast and/or osteoclast function.

Arthralgia and Osteoporosis Treatments

In one embodiment, the disclosure provides pharmaceutical compositions which are particularly useful for treating arthralgia or osteoporosis in an individual infected with Zika virus (ZIKV). In some embodiments, a treatment is provided which reduces the expression or activity of IFN gamma. The treatment thus provides one or more compounds that inhibit IFN gamma. In some embodiments, the inhibitor is an anti-IFN gamma antibody or antigen binding fragment thereof. Suitable antibodies are known to the skilled person and examples in human treatment are also described in EP1975180. In some embodiments the inhibitor is a nucleic acid molecule that binds IFN gamma mRNA or pre-mRNA, preferably said inhibitor is an antisense oligonucleotide, miRNA, or siRNA. In some embodiments, the inhibitor is a glucocorticoid. Glucocorticoids, in particular dexamethasone, are known to inhibit IFN-gamma signaling (see, e.g., Hu et al. J Immunol. 2003 70:4833-9). In another embodiment the treatment reduces type I interferon signaling. Glucocorticoids may be used to reduce type I interferon signaling.

In some embodiments, a treatment is provided which increases the expression or activity of alkaline phosphatase (ALP). Reduced enzymatic activity of ALP is reported to lead to hypophosphatasia in combination with hypercalcemia and skeletal defects (Mochizuki 2000 Eur J Pediatr). Restoring the enzymatic activity or expression of ALP may be a potential strategy to restore healthy bone development or homeostasis. Preferably wherein the one or more compounds is an ALP enzyme replacement therapy such as asfotase alfa (Strensiq™). In some embodiments, the treatment comprises providing an individual with ALP or a nucleic acid encoding ALP. Exemplary human protein and mRNA sequences of ALP are described herein.

In some embodiments, a treatment is provided which increases the expression or activity of osteocalcin. Osteocalcin is a protein that is secreted by osteoblasts and regulates bone remodeling and energy metabolism. The protein comprises a Gla domain, which can bind to calcium and hydroxyapatite. Osteocalcin levels are used as an index for active bone turnover. Increasing the expression levels of Osteocalcin may be a potential strategy to restore healthy bone development or homeostasis. In some embodiments, the treatment comprises providing an individual with osteocalcin or a nucleic acid encoding osteocalcin. Exemplary human protein and mRNA sequences of osteocalcin are described herein.

In some embodiments, a treatment is provided which reduces the RANKL/OPG ratio. The treatment thus provides one or more compounds that inhibit RANKL. In some embodiments, the inhibitor is an anti-RANKL antibody or antigen binding fragment thereof. Preferably the antibody is denosumab. In some embodiments the inhibitor is a nucleic acid molecule that binds RANKL mRNA or pre-mRNA, preferably said inhibitor is an antisense oligonucleotide, miRNA, or siRNA.

In some embodiments, the treatment comprises providing an individual with OPG or a nucleic acid encoding OPG. Exemplary human protein and mRNA sequences of OPG are described herein.

In some embodiments, a treatment is provided which reduces the expression or activity of IL-6. The treatment thus provides one or more compounds that inhibit IL-6. In some embodiments, the inhibitor is an anti-IL-6 antibody or antigen binding fragment thereof. Preferably the antibody is siltuximab. Preferably the antibody is olokizumab. Preferably the antibody is elsilimomab. Preferably the antibody is clazakizumab. Preferably the antibody is sirukumab. In some embodiments the inhibitor is a nucleic acid molecule that binds IL-6 mRNA or pre-mRNA, preferably said inhibitor is an antisense oligonucleotide, miRNA, or siRNA.

In some embodiments, the inhibitor is an anti-IL-6 receptor antibody or antigen binding fragment thereof. Preferably the antibody is tocilizumab. Preferably the antibody is Sarilumab.

In some embodiments, a treatment is provided which reduces the expression or activity of IL-1beta. The treatment thus provides one or more compounds that inhibit IL-1beta. In some embodiments, the inhibitor is an anti-IL-1beta; antibody or antigen binding fragment thereof. Preferably the antibody is canakinumab. In some embodiments the inhibitor is a nucleic acid molecule that binds IL-1beta mRNA or pre-mRNA, preferably said inhibitor is an antisense oligonucleotide, miRNA, or siRNA.

In some embodiments the inhibitor of IL-1 signaling is a IL-1 receptor antagonist, preferably anakinra. In some embodiments the inhibitor of IL-1 signaling is a soluble decoy receptor, preferably rilonacept.

In some embodiments, a treatment is provided which increases the expression or activity of the Fibroblast Growth Factor Receptor pathway. In some embodiments, the treatment comprises providing an individual with FGFR2 or a nucleic acid encoding FGFR2. Exemplary human protein and mRNA sequences of FGFR2 are described herein. In some embodiments, the treatment comprises activating the FGFR pathway with ligands for the FGFR2 receptor. For example fibroblastic growth factors (FGF), such as FGF1 and FGF2. In some embodiments, the treatment comprises activating the FGFR2 receptor by mediating receptor dimerization, for example by antibodies that stimulate the dimerization.

In some embodiments, a treatment is provided which increases the expression or activity of the BMP signaling pathway. In some embodiments, the treatment comprises providing an individual with BMP2, BMP4, follistatin or a nucleic acid encoding BMP2, BMP4, or follistatin. Exemplary human protein and mRNA sequences of BMP4, are described herein. In some embodiments, the treatment comprises activating the BMP pathway with small molecule activators, for example isoliquiritigenin, 4′-hydroxychalcone, apigenin and/or diosmetin (Vrijens et al. 2013 Plos one) or Ventromorphins (Genthe et al. 2017 ACS Chem Biol).

In some embodiments, a treatment is provided which increases the expression or activity of the Wnt signaling pathway. In some embodiments, the treatment comprises providing an individual with a Wnt (preferably Wnt3a or Wnt5), or a nucleic acid encoding a Wnt. In some embodiments, the treatment comprises providing CHIR99021, to inhibit the kinase GSK3. In some embodiments, the treatment comprises providing lithium chloride. In some embodiments, the treatment comprises providing Wnt agonist 1.

An exemplary human sequence of WNT3A is as follows:

MAPLGYFLLLCSLKQALGSYPIWWSLAVGPQYSSLGSQPILCASIPGLVP
KQLRFCRNYVEIMPSVAEGIKIGIQECQHQFRGRRWNCTTVHDSLAIFGP
VLDKATRESAFVHAIASAGVAFAVTRSCAEGTAAICGCSSRHQGSPGKGW
KWGGCSEDIEFGGMVSREFADARENRPDARSAMNRHNNEAGRQAIASHMH
LKCKCHGLSGSCEVKTCWWSQPDFRAIGDFLKDKYDSASEMVVEKHRESR
GWVETLRPRYTYFKVPTERDLVYYEASPNFCEPNPETGSFGTRDRTCNVS
SHGIDGCDLLCCGRGHNARAERRREKCRCVFHWCCYVSCQECTRVYDVHT
CK

The corresponding mRNA sequence may be found in GenBank as NCBI Reference Sequence: NM_033131.3 (23 Apr. 2018).

An exemplary human sequence of WNT5A is as follows:

MKKSIGILSPGVALGMAGSAMSSKFFLVALAIFFSFAQWIEANSWWSLGM
NNPVQMSEVYIIGAQPLCSQLAGLSQGQKKLCHLYQDHMQYIGEGAKTGI
KECQYQFRHRRWNCSTVDNTSVFGRVMQIGSRETAFTYAVSAAGWNAMSR
ACREGELSTCGCSRAARPKDLPRDWLWGGCGDNIDYGYRFAKEFVDARER
ERIHAKGSYESARILMNLHNNEAGRRTVYNLADVACKCHGVSGSCSLKTC
WLQLADFRKVGDALKEKYDSAAAMRLNSRGKLVQVNSRFNSPTTQDLVYI
DPSPDYCVRNESTGSLGTQGRLCNKTSEGMDGCELMCCGRGYDQFKTVQT
ERCHCKFHWCCYVKCKKCTEIVDQFVCK

The corresponding mRNA sequence may be found in GenBank as NCBI Reference Sequence: NM_003392.4 (7 May 2018).

An exemplary human sequence of WNT1 is as follows:

MGLWALLPGWVSATLLLALAALPAALAANSSGRWWGIVNVASSTNLLTDS
KSLQLVLEPSLQLLSRKQRRLIRQNPGILHSVSGGLQSAVRECKWQFRNR
RWNCPTAPGPHLFGKIVNRGCRETAFIFAITSAGVTHSVARSCSEGSIES
CTCDYRRRGPGGPDWHWGGCSDNIDFGRLFGREFVDSGEKGRDLRFLMNL
HNNEAGRTTVFSEMRQECKCHGMSGSCTVRTCWMRLPTLRAVGDVLRDRF
DGASRVLYGNRGSNRASRAELLRLEPEDPAHKPPSPHDLVYFEKSPNFCT
YSGRLGTAGTAGRACNSSSPALDGCELLCCGRGHRTRTQRVTERCNCTFH
WCCHVSCRNCTHTRVLHECL

The corresponding mRNA sequence may be found in GenBank as NCBI Reference Sequence: NM_005430.3 (29 Mar. 2018).

In some embodiments, a bone anabolic therapy is provided. In some embodiments, the bone anabolic therapy comprises providing Sclerostin (SOST) or a nucleic acid molecule encoding Sclerostin.

An exemplary human sequence of SOST is as follows

MQLPLALCLVCLLVHTAFRVVEGQGWQAFKNDATEIIPELGEYPEPPPEL
ENNKTMNRAENGGRPPHHPFETKDVSEYSCRELHFTRYVTDGPCRSAKPV
TELVCSGQCGPARLLPNAIGRGKWWRPSGPDFRCIPDRYRAQRVQLLCPG
GEAPRARKVRLVASCKCKRLTRFHNQSELKDFGTEAARPQKGRKPRPRAR
SAKANQAELENAY

The corresponding mRNA sequence may be found in GenBank as NCBI Reference Sequence: NM_025237.2 (16 Apr. 2018).

Other suitable anabolic therapies are well-known to a skilled person and include growth hormone, insulin-like growth factor (IGF) 1, strontium, fluoride, bone morphogenetic protein (BMP)-2, BMP-7 (i.e., osteogenic protein-1 [OP-1]), basic fibroblast growth factors (bFGFs) in particular FGF-2, and vascular endothelial growth factor (VEGF). See Lane and Kelman for a review of bone anabolic therapies (Arthritis Res Ther. 2003; 5(5): 214-222. Exemplary treatments include the short term or intermittent use of parathyroid hormone (PTH). Recombinant human parathyroid hormone (rhPTH) increases bone mass and strength and has been used to treat osteoporosis.

In some embodiments, an osteoclast activity inhibitor is provided. In some embodiments the osteoclast activity inhibitor is a bisphosphonate or denosumab.

Craniosynostosis Treatments

In one embodiment, the disclosure provides pharmaceutical compositions which are particularly useful for treating craniosynostosis in an individual infected with Zika virus (ZIKV). As is clear to a skilled person, the compounds and compositions provided herein may be administered to the individual or to a subject carrying the individual (fetus), such as a gestating mother.

In some embodiments, a treatment is provided which increases the expression or activity of IFNγ. In some embodiments, the treatment comprises providing an individual with IFNγ or a nucleic acid encoding IFNγ. Exemplary human protein and mRNA sequences of IFNγ are described herein. Human recombinant IFN-gamma is also commercially available (e.g., STEMCELL™ Technologies). The treatment also includes IFN-gamma variants, such as those described in U.S. Pat. No. 604,603 having increased stability.

In some embodiments, a treatment is provided which reduces the expression or activity of alkaline phosphatase (ALP). The treatment thus provides one or more compounds that inhibit ALP. In some embodiments, the inhibitor is an anti-ALP antibody or antigen binding fragment thereof. In some embodiments the inhibitor is a nucleic acid molecule that binds ALP mRNA or pre-mRNA, preferably said inhibitor is an antisense oligonucleotide, miRNA, or siRNA.

In some embodiments, a treatment is provided which reduces the expression or activity of osteocalcin. The treatment thus provides one or more compounds that inhibit osteocalcin. In some embodiments, the inhibitor is an anti-osteocalcin antibody or antigen binding fragment thereof. In some embodiments the inhibitor is a nucleic acid molecule that binds osteocalcin mRNA or pre-mRNA, preferably said inhibitor is an antisense oligonucleotide, miRNA, or siRNA.

In some embodiments, a treatment is provided which increases the RANKL/OPG ratio. In some embodiments, the treatment comprises providing an individual with RANKL or a nucleic acid encoding RANKL. Exemplary human protein and mRNA sequences of RANKL are described herein. In some embodiments, the treatment comprises providing an individual with glucocorticoids. Glucocorticoids affect bone remodeling and increase RANKL expression (Francisco J. A. De Paula, et al. in Williams Textbook of Endocrinology (Thirteenth Edition), Osteoporosis and Bone Biology 2016)

In some embodiments, a treatment is provided which reduces the expression or activity of OPG. The treatment thus provides one or more compounds that inhibit OPG. In some embodiments, the inhibitor is an anti-OPG antibody or antigen binding fragment thereof. In some embodiments the inhibitor is a nucleic acid molecule that binds OPG mRNA or pre-mRNA, preferably said inhibitor is an antisense oligonucleotide, miRNA, or siRNA.

In some embodiments, a treatment is provided which reduces the expression or activity of the Fibroblast Growth Factor Receptor pathway. The treatment thus provides one or more compounds that inhibit the pathway. In some embodiments, the inhibitor is an anti-FGFR2 antibody or antigen binding fragment thereof. For example, HuGAL-FR21 is a humanized anti-FGFR2. In some embodiments the inhibitor is a nucleic acid molecule that binds FGFR2 mRNA or pre-mRNA, preferably said inhibitor is an antisense oligonucleotide, miRNA, or siRNA. In some embodiments, the inhibitor is an “FGF ligand trap” which is able to bind and sequester FGFs (see Presta et al. Pharmacol Ther. 2017 November; 179:171-187 for a review of FGF ligand traps).

In some embodiments, a treatment is provided which reduces the expression or activity of the BMP signaling pathway. The treatment thus provides one or more compounds that inhibit the pathway. In some embodiments, the inhibitor is an anti-BMP2, BMP4, or follistatin antibody or antigen binding fragment thereof. In some embodiments the inhibitor is a nucleic acid molecule that binds BMP2, BMP4, or follistatin mRNA or pre-mRNA, preferably said inhibitor is an antisense oligonucleotide, miRNA, or siRNA. Preferably the inhibitor is a small molecule inhibitor of BMP signaling, preferably selected from K02288, LDN-193189, and dorsomorphin (see, e.g., Sanvitale et al., 2013 PLoS ONE 8(4):e62721).

In some embodiments the inhibitor is Noggin, Gremlin 1, Chordin or a nucleic acid molecule encoding Noggin, Gremlin 1, or Chordin.

An exemplary human sequence of Noggin is as follows

MERCPSLGVTLYALVVVLGLRATPAGGQHYLHIRPAPSDNLPLVDLIEHP
DPIFDPKEKDLNETLLRSLLGGHYDPGFMATSPPEDRPGGGGGAAGGAED
LAELDQLLRQRPSGAMPSEIKGLEFSEGLAQGKKQRLSKKLRRKLQMWLW
SQTFCPVLYAWNDLGSRFWPRYVKVGSCFSKRSCSVPEGMVCKPSKSVHL
TVLRWRCQRRGGQRCGWIPIQYPIISECKCSC (signal peptide
underlined)

The corresponding mRNA sequence may be found in GenBank as NCBI Reference Sequence: NM_005450.4 (23 Apr. 2018).

An exemplary human sequence of Gremlin-1 is as follows

MSRTAYTVGALLLLLGTLLPAAEGKKKGSQGAIPPPDKAQHNDSEQTQSP
QQPGSRNRGRGQGRGTAMPGEEVLESSQEALHVTERKYLKRDWCKTQPLK
QTIHEEGCNSRTIINRFCYGQCNSFYIPRHIRKEEGSFQSCSFCKPKKFT
TMMVTLNCPELQPPTKKKRVTRVKQCRCISIDLD

The corresponding mRNA sequence may be found in GenBank as NCBI Reference Sequence: NM_060565.1 (28 Mar. 2018).

An exemplary human sequence of Chordin is as follows

MPSLPAPPAPLLLLGLLLLGSRPARGAGPEPPVLPIRSEKEPLPVRGAAG
CTFGGKVYALDETWHPDLGEPFGVMRCVLCACEAPQWGRRTRGPGRVSCK
NIKPECPTPACGQPRQLPGHCCQTCPQERSSSERQPSGLSFEYPRDPEHR
SYSDRGEPGAEERARGDGHTDFVALLTGPRSQAVARARVSLLRSSLRFSI
SYRRLDRPTRIRFSDSNGSVLFEHPAAPTQDGLVCGVWRAVPRLSLRLLR
AEQLHVALVTLTHPSGEVWGPLIRHRALAAETFSAILTLEGPPQQGVGGI
TLLTLSDTEDSLHFLLLFRGLLEPRSGGLTQVPLRLQILHQGQLLRELQA
NVSAQEPGFAEVLPNLTVQEMDWLVLGELQMALEWAGRPGLRISGHIAAR
KSCDVLQSVLCGADALIPVQTGAAGSASLTLLGNGSLIYQVQWGTSSEVV
AMTLETKPQRRDQRTVLCHMAGLQPGGHTAVGICPGLGARGAHMLLQNEL
FLNVGTKDFPDGELRGHVAALPYCGHSARHDTLPVPLAGALVLPPVKSQA
AGHAWLSLDTHCHLHYEVLLAGLGGSEQGTVTAHLLGPPGTPGPRRLLKG
FYGSEAQGVVKDLEPELLRHLAKGMASLMITTKGSPRGELRGQVHIANQC
EVGGLRLEAAGAEGVRALGAPDTASAAPPVVPGLPALAPAKPGGPGRPRD
PNTCFFEGQQRPHGARWAPNYDPLCSLCTCQRRTVICDPWCPPPSCPHPV
QAPDQCCPVCPEKQDVRDLPGLPRSRDPGEGCYFDGDRSWRAAGTRWHPV
VPPFGLIKCAVCTCKGGTGEVHCEKVQCPRLACAQPVRVNPTDCCKQCPV
GSGAHPQLGDPMQADGPRGCRFAGQWFPESQSWHPSVPPFGEMSCITCRC
GAGVPHCERDDCSLPLSCGSGKESRCCSRCTAHRRPAPETRTDPELEKEA
EGS (signal peptide underlined)

The corresponding mRNA sequence may be found in GenBank as NCBI Reference Sequence: NM_003741.3 (3 Oct. 2017).

In some embodiments, a treatment is provided which reduces the expression or activity of the Wnt pathway. The treatment thus provides one or more compounds that inhibit the pathway. In some embodiments, the inhibitor is an antibody against Wnt3A, Wnt5, or Wnt agonist 1, or antigen binding fragment thereof. In some embodiments the inhibitor is a nucleic acid molecule that binds Wnt3A, Wnt5, or Wnt agonist 1 mRNA or pre-mRNA, preferably said inhibitor is an antisense oligonucleotide, miRNA, or siRNA. In some embodiments the inhibitor is Dkk1 or Sclerostin or a nucleic acid molecule encoding Dkk1 or Sclerostin.

An exemplary human sequence of Dkk1 is as follows

MMALGAAGATRVFVAMVAAALGGHPLLGVSATLNSVLNSNAIKNLPPPLG
GAAGHPGSAVSAAPGILYPGGNKYQTIDNYQPYPCAEDEECGTDEYCASP
TRGGDAGVQICLACRKRRKRCMRHAMCCPGNYCKNGICVSSDQNHFRGEI
EETITESFGNDHSTLDGYSRRTTLSSKMYHTKGQEGSVCLRSSDCASGLC
CARHFWSKICKPVLKEGQVCTKHRRKGSHGLEIFQRCYCGEGLSCRIQKD
HHQASNSSRLHTCQRH

The corresponding mRNA sequence may be found in GenBank as NCBI Reference Sequence: AY359005.1 (3 Oct. 2003).

In some embodiments, the treatment stimulates bone turnover. Exemplary treatments include chronic administration of parathyroid hormone (PTH), which leads to bone resorption.

In some embodiments disclosed herein, the treatments increase the activity or expression of a protein or a signaling pathway. In some embodiments, this is achieved by providing a pharmaceutical composition comprises a protein as disclosed herein. Proteins for therapeutic use may be isolated and purified from natural sources. Alternatively, such proteins may be recombinantly expressed using molecular cloning techniques known to a skilled person. The proteins may comprise amino acid substitutions, e.g., to enable soluble expression in host systems, to optimize protein stability, and/or to modulate immunogenicity. Said proteins may also comprise epitope or purification tags or be fused to other therapeutic proteins or proteins such as Fc or serum albumin for pharmacokinetic purposes. Preferably, the proteins have an amino acid sequence having at least 90, at least 95, or at least 99% identity to the sequences disclosed herein. Preferably, the protein source is matched to the species of the individual, or rather, a human protein is used when treating a human.

Nucleic acids encoding the proteins disclosed herein are also provided. The nucleic acids may be operably linked to additional sequences such as promoter sequences, ribosomal binding sites, transcriptional start and stop sequences, translational start and stop sequences, and enhancer or activator sequences. Promoter sequences encode either constitutive or inducible promoters.

Vectors comprising said nucleic acids are also provided. A “vector” is a recombinant nucleic acid construct, such as plasmid, phase genome, virus genome, cosmid, or artificial chromosome, to which another DNA segment may be attached. The term “vector” includes both viral and nonviral means for introducing the nucleic acid into a cell in vitro, ex vivo or in vivo. Non-viral vectors include plasmids, liposomes, electrically charged lipids (cytofectins), DNA-protein complexes, and biopolymers. Viral vectors include retrovirus, adeno-associated virus, pox, baculovirus, vaccinia, herpes simplex, Epstein-Barr and adenovirus vectors. Vector sequences may also contain one or more regulatory regions, and/or selectable markers useful in selecting, measuring, and monitoring nucleic acid transfer results.

Cells comprising said nucleic acids or vectors are also provided. The method of introduction is largely dictated by the targeted cell type include, e.g., CaPO4 precipitation, liposome fusion, lipofectin, electroporation, dextran-mediated transfection, calcium phosphate precipitation, polybrene mediated transfection, protoplast fusion, viral infection, encapsulation of the polynucleotide(s) in liposomes, and direct microinjection of the DNA into nuclei. The nucleic acids may stably integrate into the genome of the host cell or may exist either transiently or stably in the cytoplasm.

The proteins may be produced by culturing a host cell transformed with an expression vector containing nucleic acid encoding a protein. Appropriate host cells include yeast, bacteria, archaebacteria, fungi, and insect and animal cells, including mammalian cells. Preferably, the proteins are expressed in mammalian cells. Mammalian expression systems are also known in the art, and include retroviral systems.

The nucleic acid encoding the protein may also be used in gene therapy. In gene therapy applications, genes are introduced into cells in order to achieve in vivo synthesis of a therapeutically effective genetic product. “Gene therapy” includes both conventional gene therapy, where a lasting effect is achieved by a single treatment, and the administration of gene therapeutic agents, which involves the one time or repeated administration of a therapeutically effective DNA or mRNA. The nucleic acid molecule is preferably provided in a viral vector suitable for gene therapy. Viral vectors include lentivirus, retrovirus, adeno-associated virus (AAV), pox, baculovirus, vaccinia, herpes simplex, Epstein-Barr and adenovirus vectors. Appropriate vectors and delivery methods are known to a skilled person and are described, e.g., in Schlachetzki et al. Neurology, Gene therapy of the brain. (2004) 62:1275-1281 and Richardson et al. Neurosurg Clin N Am. 2009 20(2):205-10.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11:205-210 (1993)).

In some embodiments, the treatments decrease the activity or expression of a biomarker or a signaling pathway. The treatment may thus comprise providing one or more inhibitors of activity and/or expression. The term “inhibitor” is used in the broadest sense, and includes, e.g., any molecule that partially or fully blocks, inhibits, or neutralizes a biological activity of a biomarker or reduces expression of biomarker mRNA or functional protein. Suitable inhibitors include molecules that specifically bind said proteins and inhibit their respective functions or lead to their degradation (e.g., antibodies, small chemical compounds) as well as compounds that affect mRNA transcription, mRNA splicing, or protein translation (e.g., antisense oligonucleotides and siRNA). Inhibitors include polypeptides, small molecules, and nucleic acid based inhibitors.

Preferably the inhibitor is an antibody, in particular an antagonistic or “neutralizing” antibody. As used herein, the term “antibody” includes, for example, both naturally occurring and non-naturally occurring antibodies, polyclonal and monoclonal antibodies, chimeric antibodies and wholly synthetic antibodies and fragments thereof, such as, for example, the Fab′, F(ab′)2, Fv or Fab fragments, or other antigen recognizing immunoglobulin fragments. Methods of making antibodies are well known in the art and many suitable antibodies are commercially available. Preferably, the antibodies disclosed herein include antigen binding fragments (e.g., Fab′, F(ab′)2, Fv or Fab fragments).

Preferably the inhibitor is a nucleic acid molecule, in particular a molecule that causes the degradation of or inhibits the function, transcription, or translation of the biomarker in a sequence-specific manner. Exemplary nucleic acid molecules include aptamers, siRNA, artificial microRNA, interfering RNA or RNAi, dsRNA, ribozymes, antisense oligonucleotides, and DNA expression cassettes encoding said nucleic acid molecules.

In some embodiments, the nucleic acid molecule is an antisense oligonucleotide. Antisense oligonucleotides (ASO) generally inhibit their target by binding target mRNA and sterically blocking expression by obstructing the ribosome. ASOs can also be used for “exon-skipping”. ASOs can also inhibit their target by binding target mRNA thus forming a DNA-RNA hybrid that can be a substance for RNase H. In some embodiments, the nucleic acid molecule is an RNAi molecule, i.e., RNA interference molecule. Preferred RNAi molecules include siRNA, shRNA, and artificial miRNA. Methods for designing and producing suitable nucleic acid inhibitors are well-known in the art. See, e.g., Crooke et al. “RNA-Targeted Therapeutics” 2018 Cell Metabolism 27:714-739 for a review on nucleic acid inhibitors.

The nucleic acid molecule inhibitors may be chemically synthesized and provided directly to cells of interest. The nucleic acid compound may be provided to a cell as part of a gene delivery vehicle. Such a vehicle is preferably a liposome or a viral gene delivery vehicle. Liposomes are well known in the art and many variants are available for gene transfer purposes.

The pharmaceutical compositions described herein preferably comprise one or more pharmaceutically acceptable carriers, preservative, solubilizers, diluents and/or excipients and the like. Suitable pharmaceutically acceptable carriers, preservative, solubilizers, diluents and/or excipients may for instance be found in Remington: The Science and Practice of Pharmacy, 20th Edition. Baltimore, Md.: Lippincott Williams & Wilkins, 2000.

When administering the pharmaceutical compositions to an individual, the one ore more compounds are preferably dissolved in a solution that is compatible with the delivery method. For intravenous, subcutaneous, intramuscular, intrathecal and/or intraventricular administration it is preferred that the solution is a physiological salt solution. Preferred are excipients capable of forming complexes, vesicles and/or liposomes that deliver such a compound as defined herein in a vesicle or liposome through a cell membrane. Suitable excipients are known in the art.

Actual dosage levels of the pharmaceutical compositions may be varied so as to obtain an amount of the active ingredient which is effective to achieve the desired therapeutic response for a particular individual, while avoiding/minimizing toxicity. The selected dosage level will depend upon a variety of factors including the activity of the particular compound, the route of administration, the time of administration, the rate of excretion of the particular compound being employed, the duration of the treatment, other drugs, compounds and/or materials used in combination, the age, sex, weight, condition, general health and prior medical history of the patient being treated, and like factors well known in the medical arts. A physician or veterinarian having ordinary skill in the art can readily determine and prescribe the effective amount of the pharmaceutical composition required.

In some embodiments, a treatment as disclosed herein comprises a combination therapy. In some embodiments, two are more compounds are provided as part of the treatments. The compounds may be administered sequentially or simultaneously or the treatment may comprise, e.g., a first treatment with a first compound (e.g., spanning several days or weeks) and a second treatment with a second compound. In some embodiments, the two or more compounds are formulated in a single pharmaceutical composition.

In some embodiments, the therapeutic treatments disclosed herein further comprise the monitoring of said treatment using the biomarkers disclosed herein. The expression and/or activity of one or more biomarkers can be compared to a reference value as described herein or to the level previously determined in said individual (e.g., before treatment began). It is within the purview of a skilled person to determine based on biomarker expression/activity whether an individual is responding to treatment. In some embodiments, biomarkers are provided, wherein an increase in expression/activity indicates poor prognosis. A significant decrease in the expression/activity level in the sample as compared to a previous level is said individual, indicates that the individual is responding to treatment. In some embodiments, biomarkers are provided, wherein a decrease in expression/activity indicates poor prognosis. A significant increase in the expression/activity level in the sample as compared to a previous level is said individual, indicates that the individual is responding to treatment.

### Definitions

“Individual” as used herein includes, but is not limited to, mammals, including, e.g., a human, a non-human primate, a mouse, a pig, a cow, a goat, a cat, a rabbit, a rat, a guinea pig, a hamster, a degu, a horse, a monkey, a sheep. Preferably, the individual is a human. In some embodiments the individual in a human fetus. Preferably, the subject carrying a fetus (i.e., the individual) is a gestating mother.

A “significant” alteration in a value, as used herein, can refer to a difference which is reproducible or statistically significant, as determined using statistical methods that are appropriate and well-known in the art, generally with a probability value of less than five percent chance of the change being due to random variation. Preferably, a significant increase is at least 20, at least 40, or at least 50% higher than the reference value. It is well within the purview of a skilled person to determine the amount of increase or similarity that is considered significant.

As used herein, “to comprise” and its conjugations is used in its non-limiting sense to mean that items following the word are included, but items not specifically mentioned are not excluded. In addition the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. As used herein, treating an individual infected with ZIKV, preferably refers to treating, preventing, slowing the progression, and/or reducing one or more symptoms of the infection. In particular, the one or more symptoms are related bone metabolism, such as arthralgia, osteoporosis, and craniosynostosis.

All patent and literature references cited in the present specification are hereby incorporated by reference in their entirety.

The invention is further explained in the following examples. These examples do not limit the scope of the invention, but merely serve to clarify the invention.

### EXAMPLES

### Example 1A

Materials and Methods:

Primary cell cultures: Human—bone marrow derived mesenchymal stromal cells (hMSCs) of two healthy donors were purchased from Lonza (PT-2501). hMSCs were differentiated into osteoblast as as described previously (15). Briefly, hMSCs were cultured in αMEM medium (Gibco, Thermofisher) supplemented with 10% heat-inactivated fetal calf serum a (FCS, Sigma), 20 mM Hepes (Sigma), streptomycin/penicillin (Thermofisher) and 1.8 mM CaCl₂ (Sigma) at 37° C. and 5% CO₂ in a humidified atmosphere. Medium on day 3 post seeding (d=−3) was supplemented with 100 mM dexamethasone (dex) and 10 mM 6-glycerophosphate for differentiation into mineralizing osteoblasts within 2-3 weeks. The media were refreshed twice per week. Vero cells (African green monkey kidney epithelial cells, ATCC CCL-81) were cultured in Dulbecco's modified Eagle's medium (DMEM, Lonza, the Netherlands) supplemented with 10% heat inactivated fetal bovine serum (FBS, Greiner Bio-One, Austria), 2 mM L-glutamin (Lonza), 1% sodium bicarbonate (Lonza), 1% Hepes (Lonza), 100 U/mL penicillin (Lonza), 100 μg/mL streptomycin (Lonza) at 37° C. and 5% CO₂ in a humidified atmosphere.

Virus: Zika virus Suriname ZIKVNL00013 (ZIKVAS-Sur16) was isolated from a patient in The Netherlands (EVAg no. 011V-01621) (12). Virus stock used in this study was grown in Vero cells and passage number 3 was used for current study. Virus titer in the supernatant was determined by endpoint titrations on Vero cells as described previously (16). Briefly, tenfold serial dilution were made and inoculated onto a monolayer of Vero cells in 96 wells plate (2×10⁴ cells/well). Cytopathic effect (CPE) was used as read out and determined at 5 days post-infection (dpi), and virus titers were calculated as the 50% tissue culture infective dose (TCID50) using the Spearman-Kärber method (17). An initial 1:10 dilution of supernatant resulted in a detection limit of 10^{1.5}TCID50/ml.

Replication Kinetics of ZIKV

hMSCs were seeded three days prior to infection (day=−3). Three days post-seeding (day=0), pre-osteoblast cells were refreshed with medium containing osteogenic stimulants. After 6 hours of stimulation, cells were infected at a multiplicity of infection (moi) of 5 with ZIKV for 1 hour at 37° C. in 5% CO₂. After incubation, the supernatant was removed and cells were washed three times with αMEM medium containing 10% heat-inactivated FCS. αMEM medium containing 10% heat-inactivated FCS supplemented with 100 nM dexamethasone (dex) and 10 mM ß-glycerophosphate was added and cells were cultured for up to three weeks depending on experiment. Un-infected controls were treated and run parallel. To determine the ZIKV infectious titers produced, cell supernatants were collected at different time points, and after 96 hrs infected cells were fixed and stained for immunofluorescence assay (IFA) to confirm the infection. Supernatant was stored at −80 till further use. Experiments were performed in triplicate with two different donors.

Immunofluorescence Microscopy

Infected cells from the replication growth kinetics assay were fixed with 4% PFA on day 4 post infection, permeabilized with 70% ethanol and were stained for IFA as described previously (16). Briefly, cells after incubation with mouse monoclonal antibody anti-flavivirus group antigen (MAB10216) clone D1-4G2-4-15 (Millipore, Germany) were stained with goat anti-mouse IgG conjugated with Alexa Fluor 488 (Life technologies, the Netherlands). After incubation, cells were mounted in ProLong® Diamond Antifade Mountant with DAPI (Life technologies, USA). Non-infected cells and ZIKV-infected cells stained with mouse isotype IgG2a antibody (Dako, Denmark) were used as negative controls. ZIKV-infected cells were identified by use of a Zeiss LSM 700 confocal laser scanning microscope fitted on an Axio observer Z1inverted microscope (Zeiss). All images were processed using Zen 2010 software (Zeiss).

Alkaline Phosphatase, Mineralization, and Protein Assays

ALP and calcium measurements were performed as described previously (18). Briefly, ALP activity was determined by an enzymatic reaction, where the ALP-mediated conversion of para-nitrophenylphosphate (pNPP) [Sigma] to paranitrophenol (PNP) during 10 min at 37° C. is measured at 405 nm. For calcium measurements, cell lysates were incubated overnight with 0.24 M HCl at 4° C. Calcium content was determined colorimetrically using a calcium assay reagent prepared by combining 1 M ethanolamine buffer (pH 10.6) with 0.35 mM 0-cresolphthalein complex one in a ratio of 1:1. ALP results were adjusted for protein content of the cell lysates. For protein measurement, 200 μL of working reagent was added to 25 μL of cell lysate. The mixture was incubated for 30 min at 37° C., cooled down to room temperature (RT) for 10 min, and absorbance was measured at 595 nm. All measurements were performed using a Victor2 plate reader.

Quantification of mRNA Expression

ZIKV infected and uninfected cultures continuously treated with 100 mM dexamethasone, and 10 mM ß-glycerolphosphate, were harvested at different time points during the differentiation and maturation period (day 7, 14, 18/21 post infection). RNA isolation, cDNA synthesis, and PCR reactions were performed as described previously (15). Oligonucleotide primer pairs were designed to be either on exon boundaries or spanning at least one intron (Table 1). Gene expression was corrected for the housekeeping gene Glyceraldehyde 3-phosphate dehydrogenase, GAPDH.

Statistical Analysis

The statistical analyses were performed using GraphPad Prism 5.01 software. All results are expressed as means with standard error of the mean (S.E.M.). Student's t-test and Mann Whitney U test was used for the comparison between two groups (infected versus un-infected). P value ≤0.05 was considered significant.

Results:

Use of Human MSC Model for Arthralgia and Osteoporosis:

Human bone marrow-derived mesenchymal stem cells (MSCs) have proven to be an insightful bone formation model by studying molecular and cellular processes related to stem cell decision making, osteoblast differentiation and mineralization. In addition to osteoblasts, these cells can be driven towards adipocytes and chondrocytes, depending on the stimuli these stem cells are subjected to. Routinely performed assays to study osteoblast differentiation and maturation include the analyses of the enzymatic activity of alkaline phosphatase, the deposition of mineral in the extracellular matrix (ECM), marker gene expression and several stainings for mineralization (Curr Protoc Stem Cell Biol. 2011 June; Chapter 1). This clearly indicates that MSCs are pluripotent and it is this aspect that allows this model to be employed to study arthralgia and osteoporosis (see also. J Cell Physiol. 2018 Jun; 233(6):4895-4906. doi: 10.1002/jcp.26298. Epub 2018 Jan. 15). Indeed intra-articular injection of MSCs is being explored as a treatment against osteoarthritis (Am J Sports Med. 2017 October; 45(12):2774-2783. doi: 10.1177/0363546517716641. Epub 2017 Jul. 26). Infection of MSCs at Day 0 of culture therefore represents a suitable model of pluripotency to study arthralgia and osteoporosis.

Use of Human Osteoblast Model for Craniosynostosis:

When MSCs are being stimulated with the correct growth factors, the cells become committed towards osteoblasts. Ample studies have provided novel insights into the molecular mechanisms underlying the decision making process of MSCs towards osteoblasts as well as their differentiation and mineralization (Stem Cell Reports. 2017 Apr. 11; 8(4):947-960. doi: 10.1016/j.stemcr.2017.02.018. Epub 2017 Mar. 23; JBMR Plus. 2017 Apr. 28; 1(1):16-26. doi: 10.1002/jbm4.10003. eCollection 2017 Aug.; Proc Natl Acad Sci USA. 2015 Oct. 13; 112(41):12711-6. doi: 10.1073/pnas.1501597112. Epub 2015 Sep. 29). The chosen time point for our ZIKV infection to study craniosynostosis (Day 7) represents a situation where the pluripotent MSCs have been driven into a homogeneous osteoblastic genotype and phenotype representing a bone forming and mineralizing culture. This model is ideally suited to monitor changes in bone mineralization capacity, such as is the case for craniosynostosis. Craniosynostosis is typically a disease of altered mineralization by osteoblasts, leading to premature fusion of skull plates, which consist primarily of osteoblasts and not of other MSC-derived cell types (Biomed Res Int. 2013; 2013:292506. doi: 10.1155/2013/292506. Epub 2013 May 9; BMC Med Genet. 2018 May 30; 19(1):91. doi: 10.1186/s12881-018-0607-8). Therefore, our Day 7 infection model ideally reflects the situation of bone formation in the skull and allows for studying the process of craniosynostosis.

Replication Kinetics of ZIKV

In order to determine whether the osteoblasts are susceptible to infection with ZIKV, we infected pre-osteoblast cultures with ZIKV at moi of 5. The infected cultures did not show any cytopathic effect due to ZIKV infection. Intracellular ZIKV infection was confirmed by IFA at day 4 post-infection and infectious virus titer was confirmed by end point titration assay. ZIKV efficiently infected differentiating osteoblasts and produced high infectious titers of 10⁷ TCID₅₀/mL within 2 days post infection. Virus growth kinetics over the period of differentiation showed that pre-osteoblast cultures were persistently infected with ZIKV and shedding of infectious virions was observed over the period of three weeks post infection for both donors (FIG.; 1).

ALP and Mineralization Assay:

The effect of ZIKV infection on the differentiation and maturation of osteoblasts was determined after infecting the cultures at a moi of 5 (d=0), and quantifying the ALP activity and mineral contents at different time points post infection. In ZIKV infected osteoblasts, ALP activity was significantly reduced (p value<0.05) on day 11 post infection compared to uninfected controls (FIG.; 2). Additionally, ZIKV infection significantly reduced the maturation in terms of mineral contents (p value<0.05) in ZIKV infected osteoblasts compared to uninfected controls at late time points, day 18 and 21 post infection, during maturation.

mRNA Expression Analyses:

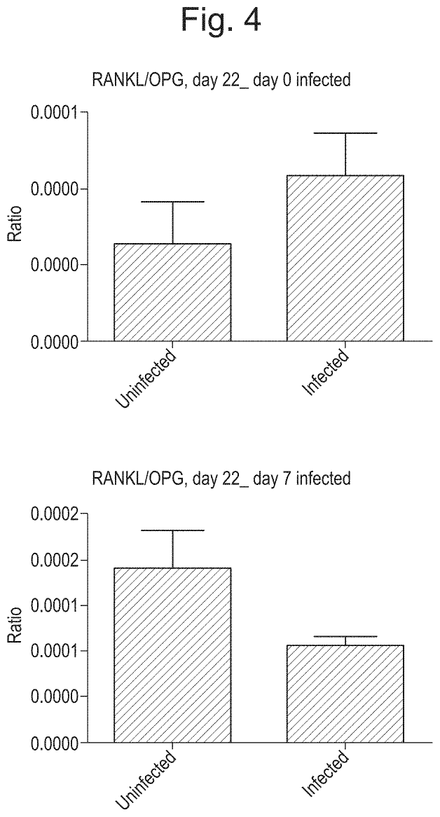

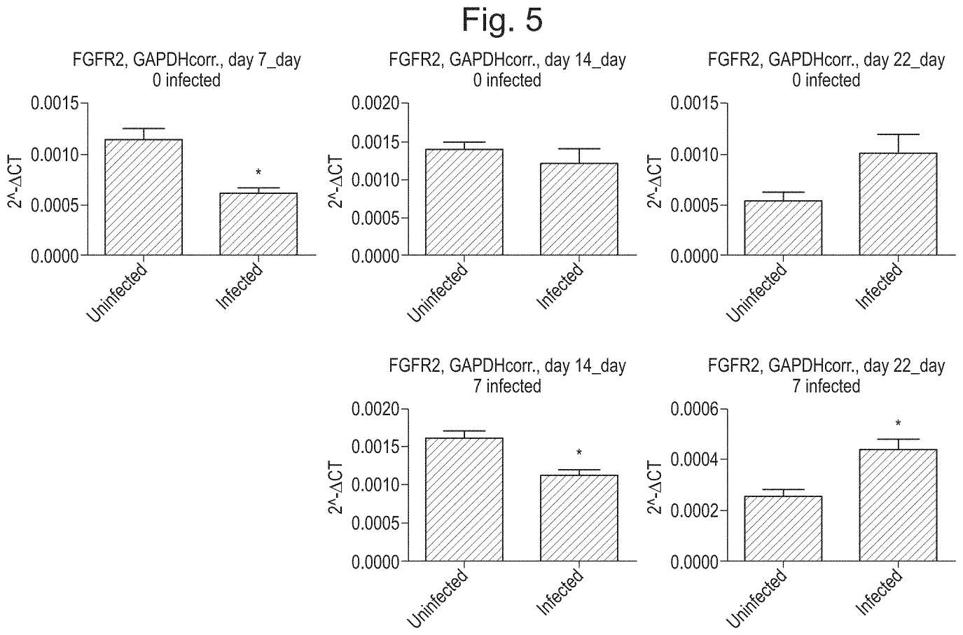

To quantify the impact of ZIKV infection on the expression levels of key transcription factors during ZIKV infection, which play an instrumental role in determining the osteoblasts phenotype, we infected osteoblasts with ZIKV at an moi of 5 on day 3 post seeding (d=0). We quantified the gene expression of key biomarkers for osteoblast proliferation, differentiation, maturation and bone homeostasis at different time points post-infection during osteoblast differentiation (see, FIG. 4 and FIG. 5). Gene expression of ALP, Runt-related transcription factor 2 (RUNX2, a key transcription factor for osteoblast differentiation) and a classical inflammatory mediator interleukin 6 (IL-6) were measured. A significant reduction of ALP and RUNX2 expression (˜2 fold) in ZIKV infected differentiating osteoblasts was observed as compared to uninfected controls (p value<0.05) (FIG.; 3). Interestingly, the levels of IL-6 were significantly (p value <0.05) increased in infected osteoblasts.

### Example 1B

Identification of Additional Markers of Prognosis:

Cell culture and total RNA isolation was identical to the experiments to generate the PCR data. Using an unbiased approach, next generation sequencing (NGS) was performed to generate gene lists and cellular pathways associated with ZIKV infection in the Day 0 (arthralgia) and Day 7 infection model (craniosynostosis). The methodology was identical to what has been extensively described previously (J Cell Biochem. 2014 October; 115(10):1816-28). Following NGS and several quality controls, the resultant gene lists were stringently filtered for expression level, significance of expression and fold-regulation by ZIKV infection compared to control conditions. Given this stringency and applied cut-offs, all genes provided in the lists are regarded as high-priority genes associated with the specifically identified pathways following ZIKV infection in both cell culture models.

Host transcriptome analysis was performed on ZIKV infected MSC's. Annotation analysis of differentially expressed mRNAs resulted in identification of the interferon signaling pathway as a top canonical pathway activated during ZIKV infection (see Table 1).

TABLE 1
Fold changes in expression of genes
of the interferon signaling pathway
following ZIKV infection in osteoblasts.

| Gene Symbol | Expr Log Ratio |
|---|---|
| MX1 | 7.87 |
| OAS1 | 6.94 |
| G1P3 | 6.62 |
| IFIT1 | 6.56 |
| G1P2 | 6.11 |
| IFIT3 | 5.00 |
| IFITM1 | 3.85 |
| IFI35 | 3.06 |
| STAT1 | 2.49 |
| TAP1 | 2.10 |
| IRF9 | 1.98 |
| PSMB8 | 1.58 |
| IFITM3 | 1.36 |
| STAT2 | 1.24 |
| IRF1 | 1.20 |

Host transcriptome analysis was performed on ZIKV infected osteoblasts. Following ZIKV infection at day 7, we have found a set of genes of the FGF signaling pathway (see Table 2), set of genes of the BMP signaling pathway (see Table 3), and a set of genes of the Wnt signaling pathway (see Tables 4 and 5) that were all strongly regulated, that point towards enhanced osteogenesis.

TABLE 2
Activation of FGF pathway during
ZIKV infection in day 7 osteoblasts.

| Gene ID | Expr Log Ratio |
|---|---|
| FGF18 | 5.746386651 |
| ERF | 4.15887107 |
| SPRY1 | 2.293644343 |
| SPRY2 | 1.794091326 |
| SPRY4 | 1.68238207 |
| FGF2 | 1.679021761 |
| FRS2 | 1.638885841 |
| FGFR1 | 1.574952205 |
| FGFR2 | 0.860409654 |
| FGFRL1 | 0.835884722 |
| THBS1 | 0.805001242 |
| FGF5 | 0.710632434 |
| FGF14 | 0.684350501 |
| FGFR3 | 0.533420967 |
| FGF7 | 0.448929347 |

TABLE 3
Activation of BMP signaling pathway during ZIKV
infection in day 7 osteoblasts. Host transcriptome
analysis was performed on ZIKV infected osteoblasts.

| Gene ID | Expr Log Ratio |
|---|---|
| AMH | 18.71960606 |
| FST | 4.390540238 |
| BMP2 | 3.219238278 |
| TGIF1 | 2.958577591 |
| BMP8B | 2.523043173 |
| TGIF2 | 2.414796733 |
| ACVR2B | 2.285210792 |
| BMP4 | 2.091935471 |
| JAG2 | 2.003232321 |
| INHBB | 1.948417506 |
| SMAD7 | 1.909490464 |
| SMAD6 | 0.872045494 |
| JAG1 | 0.857745974 |
| TGFB1I1 | 0.714338002 |
| NOG | 0.710903417 |
| BMP6 | 0.58647364 |

TABLE 4
Fold changes in expression of genes
of the Wnt/β-catenin signaling pathway
following ZIKV infection in day 7 osteoblasts.

| Gene ID | Fold change |
|---|---|
| SOX9 | 5.966976 |
| SOX4 | 4.278188 |
| SOX15 | 3.668016 |
| TLE1 | 3.080149 |
| SOX6 | 2.657372 |
| EP300 | 2.637187 |
| CREBBP | 2.475981 |
| FZD8 | 2.39828 |
| SFRP2 | 2.39828 |
| MYC | 2.096525 |
| TLE3 | 2.067661 |
| AXIN1 | 2.03637 |
| PPP2R2C | 0.495858 |

TABLE 5
Fold changes in expression of genes
of the Wnt/Ca2+ signaling pathway
following ZIKV infection in day 7 osteoblasts.

| Gene ID | Fold change |
|---|---|
| CREBBP | 2.475980582 |
| FZD8 | 2.398279828 |
| NFKB2 | 2.318191904 |
| NFATC2 | 2.267338826 |
| AXIN1 | 2.0363704 |
| PLCB4 | 0.477641468 |
| PLCE1 | 0.40332088 |

Discussion:

Following the observation that infection with ZIKV resulted in persistent or recurrent arthralgia and detection of ZIKV in synovial fluid of ZIKV infected patient, it is necessary to investigate the influence of ZIKV infection on bone remodeling and homeostasis. Currently, a biologically relevant in vitro bone model to study the ZIKV pathogenesis and subsequent osteoarticular complications is not available. Thus, in the current study we have investigated the susceptibility of osteoblasts for ZIKV infection and effects of infection on osteoblast differentiation and maturation. Current findings are in line with a recently reported study which confirms the susceptibility of human osteoblast like cell line (HOBIT) against two ZIKV strains (Asian: PRVABC59 and African: MR766) and producing 10^{7.9} TCID₅₀/mL of infectious viral titers and observed CPE over the period of 96 hrs (14). However, we have not observed any CPE in ZIKV infected primary osteoblasts for period of 3 weeks. This disparity may occur possibly due to the differences in in vitro models, as other study has used osteoblast like cell line compared to primary osteoblast cells in current study. Additionally, the presence and replication of flavivirus may not completely inhibit the host cell macromolecular synthesis which may result into non-cytopathic persistent infections (19). In the current study, the replication kinetics of ZIKV infection have shown the persistent infection of osteoblasts up to 3 weeks post infection, similar to CHIKV (7), which can be the contributing factor for viral persistence in the joints.

In both donors, ZIKV infection reduced the differentiation and maturation, in terms of ALP and mineral contents, as compared to uninfected controls which suggested that ZIKV infection had influenced the phenotype of differentiating osteoblast compared to controls. These functional assays provide clues that ZIKV infection perturbs osteoblast function, which are crucial for bone formation that can lead to bone related pathologies due to ZIKV infection. These findings were further validated by significantly reduced mRNA expression of ALP and RUNX2 during early differentiation (day 7 post infection).

In addition to bone differentiation markers, we also observed the altered levels of IL-6, a pro-inflammatory cytokines, in ZIKV infected osteoblasts. In both donors consistently high expression of IL-6 was observed. Several studies have shown that infection with arthritogenic alphaviruses, including Ross River virus (RRV) and CHIKV, results in an increase in cytokine levels particularly the levels of IL-6 and IL-ß. These key inflammatory mediators stimulate the induction of RANKL expression in osteoblast cultures. The elevated RANKL/OPG ratio leads to increased osteoclast differentiation and activation, which is previously reported to increase the bone loss and joint inflammation during RRV and CHIKV infection (5, 7). Bone remodeling is a tightly regulated process, which requires a balance in bone resorption and bone formation by osteoclasts and osteoblasts, respectively (20). Disruption of this balance can lead to abnormal bone remodeling and inception of multiple pathologic conditions. Arthritis results, in part, from pathologic bone loss due to impaired osteoblast function. Therefore, the imbalance in bone remodeling paracrine cytokines and several inflammatory cytokines such as IL-6 have been associated with arthritis (5). In the current study, the elevated levels of IL-6 in ZIKV infected osteoblasts, similar to the previous findings has highlighted the consequences of ZIKV infection in bone metabolism. In conclusion, these data clearly demonstrate that osteoblasts are susceptible to infection by ZIKV and that infection results in reduced differentiation and maturation, which can induce bone related pathologies in infected individuals.

### REFERENCES

1. Wikan N, Smith D R. Zika virus: history of a newly emerging arbovirus. The Lancet Infectious diseases. 2016; 16(7):e119-e26.
2. Lazear H M, Diamond M S. Zika virus: new clinical syndromes and its emergence in the western hemisphere. Journal of virology. 2016; 90(10):4864-75.
3. Martines R B, Bhatnagar J, de Oliveira Ramos A M, Davi H P F, Iglezias S D A, Kanamura C T, et al. Pathology of congenital Zika syndrome in Brazil: a case series. The Lancet. 2016; 388(10047):898-904.
4. Edupuganti S, Natrajan M S, Rouphael N, Lai L, Xu Y, Feldhammer M, et al., editors. Biphasic Zika illness with rash and joint pain. Open forum infectious diseases; 2017: Oxford University Press.
5. Chen W, Foo S-S, Rulli N E, Taylor A, Sheng K-C, Herrero L J, et al. Arthritogenic alphaviral infection perturbs osteoblast function and triggers pathologic bone loss. Proceedings of the National Academy of Sciences. 2014; 111(16):6040-5.
6. Borgherini G, Poubeau P, Jossaume A, Gouix A, Cotte L, Michault A, et al. Persistent arthralgia associated with chikungunya virus: a study of 88 adult patients on reunion island. Clinical Infectious Diseases. 2008; 47(4):469-75.
7. Noret M, Herrero L, Rulli N, Rolph M, Smith P N, Li R W, et al. Interleukin 6, RANKL, and Osteoprotegerin Expression by Chikungunya Virus-Infected Human Osteoblasts. The Journal of infectious diseases. 2012; 206(3):455-7.
8. Terrier C S-P, Gasque P. Bone responses in health and infectious diseases: A focus on osteoblasts. Journal of Infection. 2017; 75(4):281-92.
9. Jayamali W, Herath H, Kulatunga A. A young female presenting with unilateral sacroiliitis following dengue virus infection: a case report. Journal of medical case reports. 2017; 11(1):307.
10. Colombo T E, Estofolete C F, Reis A F N, da Silva N S, Aguiar M L, Cabrera E M S, et al. Clinical, laboratory and virological data from suspected ZIKV patients in an endemic arbovirus area. Journal of Clinical Virology. 2017; 96:20-5.
11. Chan J F, Choi G K, Yip C C, Cheng V C, Yuen K-Y. Zika fever and congenital Zika syndrome: an unexpected emerging arboviral disease. Journal of Infection. 2016; 72(5): 507-24.
12. van der Eijk A A, van Genderen P J, Verdijk R M, Reusken C B, Mogling R, van Kampen J J, et al. Miscarriage associated with Zika virus infection. New England Journal of Medicine. 2016; 375(10):1002-4.
13. Bayless N L, Greenberg R S, Swigut T, Wysocka J, Blish C A. Zika virus infection induces cranial neural crest cells to produce cytokines at levels detrimental for neurogenesis. Cell host & microbe. 2016; 20(4):423-8.
14. Colavita F, Musumeci G, Caglioti C. Human Osteoblast-like Cells Are Permissive for Zika Virus Replication. The Journal of rheumatology. 2018; 45(3):443-.
15. Both T, van de Peppel H J, Zillikens M C, Koedam M, van Leeuwen J P, van

Hagen P M, et al. Hydroxychloroquine decreases human MSC-derived osteoblast differentiation and mineralization in vitro. Journal of cellular and molecular medicine. 2018; 22(2):873-82.
16. Anfasa F, Siegers J Y, van der Kroeg M, Mumtaz N, Raj V S, de Vrij F M, et al. Phenotypic differences between Asian and African lineage Zika viruses in human neural progenitor cells. mSphere. 2017; 2(4):e00292-17.
17. Kärber G. Beitrag zur kollektiven Behandlung pharmakologischer Reihenversuche. Naunyn-Schmiedebergs Archiv für experimentelle pathologie and pharmakologie. 1931; 162(4):480-3.
18. Bruedigam C, Driel Mv, Koedam M, Peppel Jvd, van der Eerden B C, Eijken M, et al. Basic techniques in human mesenchymal stem cell cultures: differentiation into osteogenic and adipogenic lineages, genetic perturbations, and phenotypic analyses. Current protocols in stem cell biology. 2011:1H. 3.1-H. 3.20.
19. Bakonyi T, Lussy H, Weissenböck H, Hornyák Á, Nowotny N. In vitro host-cell susceptibility to Usutu virus. Emerging Infectious Diseases. 2005; 11(2):298.
20. Boyce B F, Xing L. Functions of RANKL/RANK/OPG in bone modeling and remodeling. Archives of biochemistry and biophysics. 2008; 473(2):139-46.

### Example 2

To demonstrate that blocking IL-6 and IL-16 during ZIKV infection of osteoblasts will result in restoration of RANKL/OPG expression levels, cultures of osteoblasts will be infected with ZIKV and prior to, or different time points after infection, these cultures will be treated with neutralizing antibodies against IL-6, IL-16 or a combination of both IL-6 and IL-16. We will determine the expression of RANKL and OPG in the supernatant.

### Example 3: Mouse Model

An established murine ZIKV model will be used to generate translational data on ZIKV-mediated skeletal pathogenesis pre- and postnatally. Moreover, we will perform primary bone marrow-derived osteoblast and osteoclast cultures

Intracerebral ZIKA infection of embryos can result in microcephaly postnatally. This model was previously used to study neural development and its role in microcephaly. Here we will focus on the alternative hypothesis that microcephaly is the result of premature cranial fusion. Newborns will be carefully examined for microcephaly and other phenotypes such as growth restrictions. At different time points post infection, we will perform extensive skull and long bone phenotyping of the fetal and newborn mice following ZIKA infection. These include microcomputed tomography (μCT) to study long bones and skull microarchitecture and histology to screen for craniosynostosis. Finally, the biomarkers disclosed herein will be measured in maternal and fetal tissues and serum at different time points post-infection.

Cranial suture fusion has been studied in mice by means of culturing posterior cranial sutures prior to fusion of the posterior frontal suture, which is facilitated by the underlying dura mater [Bradley, J. P., et al., Studies in cranial suture biology: regional dura mater determines in vitro cranial suture fusion. Plast Reconstr Surg, 1997. 100(5): p. 1091-9; discussion; 1100-2]. We will explant posterior frontal sutures with the underlying dura mater at 3 weeks of age, and culture these up to 30 days in vitro. Following ZIKV infection, we will scan these explants by μCT (Quantum Fx) by Perkin Elmer in animal facility) every 3 days to monitor the fusion process compared to non-infected explants. In addition, we will perform histology to compare the degree of posterior frontal suture fusion. After harvesting, we will perform gene expression studies to look for known signaling pathways of craniosynostosis (FGF, BMP). Similar to the experiments in Aim 1a, we will add in these cultures neutralizing antibodies or interferon signaling suppressing drugs or compounds that inhibit FGF and/or BMP signaling.

### Example 4

The consequences of lentiviral knockdown (using shRNAs) or knockout (using Crispr-Cas9) of ZIKV-affected genes will be assessed. In addition, specific drugs to block the type I and type II interferon signaling pathway in MSC- and NCC-derived osteoblasts following ZIKV-infection will be tested.

### Example 5

Bone and cartilage outcomes in adults and fetal/newborn humans within a cohort of pregnant women in the America's and Caribbean as part of the EU project ZIKAlliance will be assessed. ZIKAlliance is an interdisciplinary project with a global focus on the impact of ZIKV infection during pregnancy and the natural history of ZIKV in humans. It aims to include 10,000 participants from the America's in which ZIKV infection in mother and child will be closely monitored during and after pregnancy. Clinical samples (including serum from mother and child, and fetal tissues in case of abortion or still birth), are being collected from cases with evidence of craniosynostosis and/or microcephaly as well as ZIKV infection.

In this study, the clinical samples collected will be will be used to determine the expression/activity levels of biomarkers in mother/child pairs by comparing samples from children with or without craniosynostosis or other bone malformations and with or without ZIKV infection.

1.-25. (canceled)

26. A method for:
treating an individual infected with Zika virus (ZIKV) for arthralgia or osteoporosis, said method comprising determining the expression and/or activity level of one or more biomarkers indicative of osteoblast and/or osteoclast function in a sample, wherein perturbation of osteoblast and/or osteoclast function indicates poor prognosis of said individual and treating said individual determined to have a poor prognosis with a treatment for arthralgia or osteoporosis; or
treating an individual infected with Zika virus (ZIKV) for craniosynostosis, said method comprising determining the expression and/or activity level of one or more biomarkers indicative of osteoblast and/or osteoclast function in a sample, wherein perturbation of osteoblast and/or osteoclast function indicates poor prognosis of said individual, and treating an individual determined to have a poor prognosis with a treatment for craniosynostosis.

27. The method of claim 26, wherein the one or more biomarkers are selected from the group consisting of one or more markers of the type I interferon signaling pathway, interferon gamma, alkaline phosphatase, osteocalcin, the RANKL/OPG ratio, IL-6, IL-1 beta, CTX-I, DPD, one or more markers of the Fibroblast Growth Factor Receptor pathway, one or more markers of the BMP signaling pathway, or one or more markers of the Wnt signaling pathway.

28. The method of claim 26, wherein the method is for treating an individual infected with Zika virus (ZIKV) for arthralgia or osteoporosis and the one or more biomarkers are selected from the group consisting of one or more markers of the type I interferon signaling pathway, interferon gamma, IL-6, alkaline phosphatase, or RANKL/OPG ratio.

29. The method of claim 26, wherein the method is for treating an individual infected with Zika virus (ZIKV) for craniosynostosis and the one or more biomarkers are selected from the group consisting of alkaline phosphatase, the RANKL/OPG ratio, IL-6, one or more markers of the Fibroblast Growth Factor Receptor pathway, one or more markers of the BMP signaling pathway, or one or more markers of the Wnt signaling pathway.

30. The method of claim 26, wherein said expression and/or activity level is compared to a reference value.

31. The method of claim 26, wherein the method is for treating an individual infected with Zika virus (ZIKV) for craniosynostosis and the sample is from said individual or the individual is a fetus and the sample is from a subject carrying the fetus.

32. The method of claim 26, wherein the method further comprises obtaining a sample from said individual or, if the individual is a fetus, the sample from a subject carrying the fetus.

33. The method of claim 26, wherein the sample is a bodily fluid.

34. The method of claim 26, wherein determining the prognosis comprises predicting the severity of and/or risk of developing one or more symptoms related to bone metabolism.

35. A method for a) determining the prognosis of an individual infected with Zika virus (ZIKV), b) for predicting the severity of and/or risk of developing arthralgia or osteoporosis in an individual infected with Zika virus (ZIKV), or c) for predicting the severity of and/or risk of developing craniosynostosis in an individual infected with Zika virus (ZIKV), said method comprising determining the expression and/or activity level of one or more biomarkers indicative of osteoblast and/or osteoclast function in a sample, wherein perturbation of osteoblast and/or osteoclast function indicates poor prognosis of said individual.

36. The method of claim 35, wherein the one or more biomarkers are selected from the group consisting of one or more markers of the type I interferon signaling pathway, interferon gamma, alkaline phosphatase, osteocalcin, the RANKL/OPG ratio, IL-6, IL-1 beta, CTX-I, DPD, one or more markers of the Fibroblast Growth Factor Receptor pathway, one or more markers of the BMP signaling pathway, or one or more markers of the Wnt signaling pathway.

37. A kit suitable for determining the prognosis of an individual infected with Zika virus (ZIKV), said kit comprising at least three of the following:
one or more binding agents that bind IFNγ,
one or more binding agents that bind alkaline phosphatase (ALP) or one or more compounds for determining ALP activity,
one or more binding agents that bind RANKL and one or more binding agents that bind OPG,
one or more binding agents that bind IL-6,
one or more binding agents that bind IL-1beta,
one or more binding agents that bind CTX-I,
one or more binding agents that bind DPD,
one or more binding agents that bind a member of the Fibroblast Growth Factor Receptor pathway, preferably FGFR2,
one or more binding agents that bind a member of the BMP signaling pathway, preferably BMP4, and
one or more binding agents that bind a member of the Wnt signaling pathway.

38. The method of claim 26, wherein the expression and/or activity level of the one or more biomarkers is determined using a kit suitable for determining the prognosis of an individual infected with Zika virus (ZIKV), said kit comprising at least three of the following:
one or more binding agents that bind IFNγ,
one or more binding agents that bind alkaline phosphatase (ALP) or one or more compounds for determining ALP activity,
one or more binding agents that bind RANKL and one or more binding agents that bind OPG,
one or more binding agents that bind IL-6,
one or more binding agents that bind IL-1beta,
one or more binding agents that bind CTX-I,
one or more binding agents that bind DPD,
one or more binding agents that bind a member of the Fibroblast Growth Factor Receptor pathway, preferably FGFR2,
one or more binding agents that bind a member of the BMP signaling pathway, preferably BMP4, and
one or more binding agents that bind a member of the Wnt signaling pathway.

39. The method of claim 35, wherein the expression and/or activity level of the one or more biomarkers is determined using a kit suitable for determining the prognosis of an individual infected with Zika virus (ZIKV), said kit comprising at least three of the following:
one or more binding agents that bind IFNγ,
one or more binding agents that bind alkaline phosphatase (ALP) or one or more compounds for determining ALP activity,
one or more binding agents that bind RANKL and one or more binding agents that bind OPG,
one or more binding agents that bind IL-6,
one or more binding agents that bind IL-1beta,
one or more binding agents that bind CTX-I,
one or more binding agents that bind DPD,
one or more binding agents that bind a member of the Fibroblast Growth Factor Receptor pathway, preferably FGFR2,
one or more binding agents that bind a member of the BMP signaling pathway, preferably BMP4, and
one or more binding agents that bind a member of the Wnt signaling pathway.

40. A method for treating an individual infected with Zika virus (ZIKV) for arthralgia or osteoporosis, said method comprising administering to an individual in need thereof:
one or more compounds that reduces the expression or activity of IFNγ, preferably wherein the one or more compounds are selected from the group consisting of a neutralizing antibody against IFNγ and glucocorticoids;
one or more compounds that increases the expression or activity of alkaline phosphatase (ALP), preferably wherein the one or more compounds is an ALP enzyme replacement therapy, more preferably Asfotase alfa;
one or more compounds that reduces the RANKL/OPG ratio, preferably wherein the one or more compounds is an antibody against RANKL;
one or more compounds that reduces the expression or activity of IL-6, preferably wherein the one or more compounds is an antibody against IL-6;
one or more compounds that reduces the expression or activity of IL-1β, preferably wherein the one or more compounds is an antibody against IL-1β;
one or more compounds that increases the expression or activity of the Fibroblast Growth Factor Receptor pathway, preferably wherein the one or more compounds is FGF2 or a nucleic acid molecule encoding FGF2;
one or more compounds that increases the expression or activity of the BMP signaling pathway, preferably wherein the one or more compounds is selected from BMP2, BMP4, follistatin, and nucleic acids encoding BMP2, BMP4, and follistatin;
one or more compounds that increases the expression or activity of the Wnt signaling pathway, preferably wherein the one or more compounds is selected from Wnt (preferably Wnt3a or Wnt5), the small molecule CHIR99021, and lithium chloride;
one or more compounds that comprise a bone anabolic therapy, preferably, the bone anabolic therapy comprises providing Sclerostin (SOST) or a nucleic acid molecule encoding Sclerostin; or
one or more osteoclast activity inhibitors, preferably wherein the inhibitor is a bisphosphate; or
treating an individual infected with Zika virus (ZIKV) for craniosynostosis, said method comprising administering to an individual in need thereof:
one or more compounds that reduces the expression or activity of alkaline phosphatase (ALP)
one or more compounds that increases the RANKL/OPG ratio; preferably wherein the one or more compounds is RANKL or a nucleic acid encoding RANKL;
one or more compounds that reduces the expression or activity of Fibroblast Growth Factor Receptor pathway, preferably wherein the one or more compounds is a neutralizing FGFR antibody or FGF trap;
one or more compounds that reduces the expression or activity of the BMP signaling pathway, preferably wherein the one or more compounds is a small molecule inhibitor of BMP signaling, preferably selected from K02288, LDN-193189, and dorsomorphin;
Noggin, Gremlin 1, Chordin or a nucleic acid molecule encoding Noggin, Gremlin 1, or Chordin;
one or more compounds that reduces the expression or activity of the Wnt, preferably wherein the one or more compounds is Dkk1 or Sclerostin, or a nucleic acid encoding Dkk1 or Sclerostin;
one or more compounds that stimulate bone turnover, preferably wherein the compound is parathyroid hormone (PTH).

41. The method of claim 28, wherein the method comprises:
determining the expression of one or more of MX1, OAS1, G1P3, IFIT1, G1P2, IFIT3, IFITM1, IFI35, STAT1, TAP1, IRF9, PSMB8, IFITM3, STAT2, or IRF1;
determining the expression of interferon gamma;
determining the expression of IL-6;
determining the expression of alkaline phosphatase; and/or
determining the RANKL/OPG ratio.

42. The method of claim 29, wherein the method comprises:
determining the expression of alkaline phosphatase;
determining the RANKL/OPG ratio;
determining the expression of IL-6;
determining the expression of FGFR2, FGF18, ERF, SPRY1, SPRY2, SPRY4, FGF2, FRS2, FGFR1, FGFR2, FGFRL1, THBS1, FGF5, FGF14, FGFR3, and/or FGF7;
determining the expression of AMH, FST, BMP2, TGIF1, BMP8B, TGIF2, ACVR2B, BMP4, JAG2, INHBB, SMAD7, SMAD6, JAG1, TGFB1I1, NOG, and/or BMP6;
determining the expression of SOS9, SOX4, SOX15, TLE1, SOX6, EP300, CREBBP, FZD8, SFRP2, MYC, TLE3, AXIN1, and/or PP2RC; and/or
determining the expression of CREBBP, FZD8, NFKB2, NFATC2, AXIN1, PLCB4, and/or PLCE1.

43. The kit of claim 37, wherein determining the prognosis comprises predicting the severity of symptoms.
